SEQUENCE LISTING (67 sequences)

SEQ ID NO: 1 - protein, 17 aa, Mycobacterium tuberculosis
WDATATELNNALQNLAR

SEQ ID NO: 2 - protein, 19 aa, Mycobacterium tuberculosis
LAAAWGGSGSEAYQGVQQK

SEQ ID NO: 3 - protein, 18 aa, Mycobacterium tuberculosis
TQIDQVESTAGSLQGQWR

SEQ ID NO: 4 - protein, 15 aa, Mycobacterium tuberculosis
ADEEQQQALSSQMGF

SEQ ID NO: 5 - protein, 15 aa, Mycobacterium tuberculosis
TDAATLAQEAGNFER

SEQ ID NO: 6 - protein, 13 aa, Mycobacterium tuberculosis
GAAGTAAQAAVVR

SEQ ID NO: 7 - protein, 8 aa, Mycobacterium tuberculosis
QAGVQYSR

SEQ ID NO: 8 - protein, 10 aa, Zaire ebolavirus
QPLPAATWTD

SEQ ID NO: 9 - protein, 20 aa, M. tuberculosis complex
VQFQSGGNNSPAVYLLDGLR

SEQ ID NO: 10 - protein, 17 aa, M. tuberculosis complex
WETFLTSELPQWLSANR

SEQ ID NO: 11 - protein, 20 aa, Non tuberculosis mycobacterium
VQFQSGGNGSPAVYLLDGLR

SEQ ID NO: 12 - protein, 20 aa, Non tuberculosis mycobacterium
VQFQSGGDNSPAVYLLDGLR

SEQ ID NO: 13 - protein, 20 aa, Non tuberculosis mycobacterium
VQFQSGGDGSPAVYLLDGLR

SEQ ID NO: 14 - protein, 20 aa, Non tuberculosis mycobacterium
VQFQSGGEGSPAVYLLDGLR

SEQ ID NO: 15 - protein, 20 aa, Non tuberculosis mycobacterium
VQFQSGGPGSHAVYLLDGLR

SEQ ID NO: 16 - protein, 20 aa, Non tuberculosis mycobacterium
IQFQSGGPGSHAVYLLDGLR

SEQ ID NO: 17 - protein, 20 aa, Non tuberculosis mycobacterium
VQFQSGGPNSPAVYALDGLR

SEQ ID NO: 18 - protein, 17 aa, Non tuberculosis mycobacterium
WETFLTSELPSYLASNK

SEQ ID NO: 19 - protein, 17 aa, Non tuberculosis mycobacterium
WETFLTSELPQYLASNK

SEQ ID NO: 20 - protein, 17 aa, Non tuberculosis mycobacterium
WETFLTSELPEWLSANR

SEQ ID NO: 21 - protein, 17 aa, Non tuberculosis mycobacterium
WETFLTSELPQYLQSNK

SEQ ID NO: 22 - protein, 17 aa, Non tuberculosis mycobacterium
WETFLTSELPGYLSSQK

SEQ ID NO: 23 - protein, 17 aa, Non tuberculosis mycobacterium
WETFLTSELPEWLAANR

SEQ ID NO: 24 - protein, 17 aa, Non tuberculosis mycobacterium
WETFLTSELPSWLAANR

SEQ ID NO: 25 - protein, 17 aa, Non tuberculosis mycobacterium
WELFLTSELPGYLASQK

SEQ ID NO: 26 - protein, 21 aa, Non tuberculosis mycobacterium
DIVVEFQPGGPHAVYLLDGLR

SEQ ID NO: 27 - protein, 9 aa, Non tuberculosis mycobacterium
AVYLLDGLR

SEQ ID NO: 28 - protein, 17 aa, Non tuberculosis mycobacterium
WETFLTTELPQWLGANK

SEQ ID NO: 29 - protein, 17 aa, Non tuberculosis mycobacterium
WETFLTGELPQWLAANR

SEQ ID NO: 30 - protein, 32 aa, Ebola virus
LITQPLPAATWTDDTPTGSNGALRPGISFHPK

SEQ ID NO: 31 - protein, 32 aa, Ebola virus
LITQPLPAATWTDDTPTGPTGILRPGISFHPK

SEQ ID NO: 32 - protein, 32 aa, Ebola virus
LITQPLPAATWTDETPAVSTGTLRPGISFHPK

SEQ ID NO: 33 - protein, 32 aa, Ebola virus
LVTQPLPAATWTDETPSNLSGALRPGLSFHPK

SEQ ID NO: 34 - protein, 32 aa, Ebola virus
LITQPLPAATWTDETPAGAVNALRPGLSLHPK

SEQ ID NO: 35 - protein, 27 aa, M. tuberculosis complex
GVQQKWDATATELNNALQNLARTISEA

SEQ ID NO: 36 - protein, 27 aa, Non tuberculosis mycobacterium
AEQLKWHQSATALNEMLAALGNAVRDG

SEQ ID NO: 37 - protein, 27 aa, Non tuberculosis mycobacterium
AAWELWHCGAREVQLGLAMLARLVGQA

SEQ ID NO: 38 - protein, 27 aa, Non tuberculosis mycobacterium
AEQLKWHQSATALNEMLAALENAVRDG

SEQ ID NO: 39 - protein, 27 aa, Non tuberculosis mycobacterium
REQKIWQQTADTLLAAAKKLGGLISAA

SEQ ID NO: 40 - protein, 27 aa, Non tuberculosis mycobacterium
QVMAQWQTTQTQVEVALDDITAALGAA

SEQ ID NO: 41 - protein, 27 aa, Non tuberculosis mycobacterium
SVSRDYLVSAGEMKEALAALGGTTTAA

SEQ ID NO: 42 - protein, 27 aa, Non tuberculosis mycobacterium
GVQQKWDATAQELNNALQNLSRTISEA

SEQ ID NO: 43 - protein, 27 aa, Non tuberculosis mycobacterium
GVQQKWDATAQELNSALQNLSRTISEA

SEQ ID NO: 44 - protein, 27 aa, Non tuberculosis mycobacterium
AVQREWDNAATELNSSLQNLARTISEA

SEQ ID NO: 45 - protein, 27 aa, Non tuberculosis mycobacterium
IEQGRLNHRAQEFQLAVTDYLNAVENA

SEQ ID NO: 46 - protein, 27 aa, Non tuberculosis mycobacterium
AVQTRWDNTSNELNQALQNLAQTISEA

SEQ ID NO: 47 - protein, 27 aa, Non tuberculosis mycobacterium
AVQARWDSTSNELNLALQNLAQTISEA

SEQ ID NO: 48 - protein, 27 aa, Non tuberculosis mycobacterium
GVQQNWDSTAQELNNSLQNLARTISEA

SEQ ID NO: 49 - protein, 27 aa, Non tuberculosis mycobacterium
GVQQKWDSTAQELNNALQNLARTISEA

SEQ ID NO: 50 - protein, 27 aa, Non tuberculosis mycobacterium
QVQQQWDGASADLNALLQALAHAIHQS

SEQ ID NO: 51 - protein, 27 aa, Non tuberculosis mycobacterium
QVQQQWDSASADLNAALQALAHAIAQS

SEQ ID NO: 52 - protein, 27 aa, Non tuberculosis mycobacterium
GVQQKWDSTAQELNNALQNLARTISEA

SEQ ID NO: 53 - protein, 27 aa, Non tuberculosis mycobacterium
QVQQQWDSASADLNAVLQALAHAIHQS

SEQ ID NO: 54 - protein, 27 aa, Non tuberculosis mycobacterium
QVQQKWDATAQELNSSLQNLARTISEA

SEQ ID NO: 55 - protein, 27 aa, Non tuberculosis mycobacterium
AVQQKWDGTAQELNNSLQNLARTISEA

SEQ ID NO: 56 - protein, 25 aa, M. tuberculosis complex
MAEMKTDAATLAQEAGNFERISGDL

SEQ ID NO: 57 - protein, 25 aa, Non tuberculosis mycobacterium
MSSMRADTAAHAASAGRILSLADEL

SEQ ID NO: 58 - protein, 25 aa, Non tuberculosis mycobacterium
MSMLDAHIPQLVASQSAFSAKAALL

SEQ ID NO: 59 - protein, 25 aa, Non tuberculosis mycobacterium
MLDAHIPQLVASQSAFSAKAALLRS

SEQ ID NO: 60 - protein, 25 aa, Non tuberculosis mycobacterium
TMGLLDTNVPAMVSAGNSFGQNVAI

SEQ ID NO: 61 - protein, 25 aa, Non tuberculosis mycobacterium
MSAPLNVDPAQLHAVSGLAAEVARD

SEQ ID NO: 62 - protein, 25 aa, Non tuberculosis mycobacterium
MAEMKTDAATLAQEAGNFERISGDM

SEQ ID NO: 63 - protein, 25 aa, Non tuberculosis mycobacterium
MAQMNTDAAVLAKEAANFERISGEL

SEQ ID NO: 64 - protein, 25 aa, Non tuberculosis mycobacterium
MAAMNTDAAVLAKEAANFERISGEL

SEQ ID NO: 65 - protein, 25 aa, Non tuberculosis mycobacterium
MDADGTLSANAAAMLGFAESLRGAA

SEQ ID NO: 66 - protein, 22 aa, Non tuberculosis mycobacterium
LKNDAATLAQEAGNFERISGDL

SEQ ID NO: 67 - protein, 25 aa, Non tuberculosis mycobacterium
MAEMKTDAAALAQEAGNFERISSDL

(19) United States
(12) Patent Application Publication
(10) Pub. No.: US 2022/0162272 A1
(43) Pub. Date: May 26, 2022

(54) COMPOSITIONS AND METHODS OF DETERMINING A LEVEL OF INFECTION IN A SUBJECT

(71) Applicant: THE METHODIST HOSPITAL, Houston, TX (US)

(72) Inventors: Ye Hu, Scottsdale, AZ (US); Chang Liu, Lexington, SC (US); Xin Ma, Pearland, TX (US); Yaojun Li, Houston, TX (US)

(73) Assignee: THE METHODIST HOSPITAL, Houston, TX (US)

(21) Appl. No.: 17/541,365

(22) Filed: Dec. 3, 2021

Related U.S. Application Data
(63) Continuation of application No. 16/433,798, filed on Jun. 6, 2019.
Application No. 16/433,798 is a continuation of application No. PCT/US2018/015742, filed on Jan. 29, 2018.
(60) Provisional application No. 62/460,280, filed on Feb. 17, 2017.

(51) Int. Cl. C07K 14/35; A61K 47/61; A61K 47/64; G01N 33/569

(52) U.S. Cl. CPC C07K 14/35; A61K 47/61; B82Y 5/00; G01N 33/56972; A61K 47/6425

(57) ABSTRACT

Provided herein are nanoparticles configured for the rapid detection of disease-specific peptides from patient samples, including blood-based samples. Also provided are methods of measuring the level of an infection by isolation and quantification of disease-specific peptides from patient samples. The nanoparticles may act as a co-matrix for matrix assisted laser desorption/ionization mass spectrometry.

### CROSS-REFERENCE TO RELATED APPLICATIONS

This application is a continuation of U.S. patent application Ser. No. 16/433,798, filed Jun. 6, 2019, entitled COMPOSITIONS AND METHODS OF DETERMINING A LEVEL OF INFECTION IN A SUBJECT, which is a continuation of PCT International Application No. PCT/US2018/015742, filed Jan. 29, 2018, entitled COMPOSITIONS AND METHODS OF DETERMINING A LEVEL OF INFECTION IN A SUBJECT, which in turn claims the benefit of U.S. Provisional Patent Application No. 62/460,280, filed Feb. 17, 2017, entitled COMPOSITIONS AND METHODS OF DETERMINING A LEVEL OF ACTIVE MYCOBACTERIUM TUBERCULOSIS INFECTION IN A SUBJECT, all of which are incorporated herein by reference in their entirety.

### GOVERNMENT SUPPORT

This invention was made with government support under Grant No. R01-AI113725-01A1 and R01-AI-122932-01A1, awarded by the National Institutes of Health.

The government has certain rights in the invention.

### REFERENCE TO SEQUENCE LISTING, TABLE OR COMPUTER PROGRAM

The official copy of the Sequence Listing is submitted concurrently with the specification as an ASCII formatted text file via EFS-Web, with a file name of “Sequence Listing ST25.txt”, a creation date of Dec. 9, 2019, and a size of 19 kilobytes. The sequence listing filed via EFS-Web is part of the specification and is hereby incorporated in its entirety by reference herein.

### FIELD OF THE INVENTION

The present invention generally relates to compositions and methods for determining a level of disease in a subject; more particularly to compositions and methods for selectively identifying and targeting one or more biomarkers indicative of a infectious agent within a subject sample; and still more particularly to compositions and methods for selectively targeting and enriching identified biomarkers within the sample wherein the biomarker enrichment promotes signal enhancement of the target biomolecule during sample testing.

### BACKGROUND OF THE INVENTION

Infectious diseases are caused by infections and are communicable. Infectious diseases account for over one-quarter of all deaths worldwide. Leading pathogens cause pneumonia, HIV/AIDS, diarrhea, tuberculosis and malaria. Moreover, the rate and risk of epidemics has increased as the barriers separating human populations continue to shrink due to the ease and volume of intercontinental travel and the expansion of human activity. Recent examples include the widespread Ebola outbreak in 2014 and the Zika outbreak in South and Central America during 2015-2016.

Mycobacterium tuberculosis (Mtb), the microbe that causes tuberculosis (TB), is one species within the genus Mycobacterium. Active Mtb infections represent a significant global health threat. Unfortunately, detecting active TB cases and monitoring their responses to therapy remain fraught with challenges. The most widely-used diagnostic methods for TB rely on sputum samples.

Methods that apply microbiological techniques to sputum samples, such as acid-fast bacillus (AFB) smear microscopy and Mtb culture, suffer from moderate sensitivity, specificity and/or a long turnaround time. An alternative to microbiological techniques is the PCR-based Xpert MTB/RIF sputum assay. Xpert MTB/RIF is quicker and more specific than microbiological techniques but suffers from poor sensitivity under low bacterial loads and inability to distinguish live and nonviable Mtb contributions. Sputum samples are also difficult to obtain after symptomatic improvement and are often not diagnostically useful for extra-pulmonary TB (EPTB) cases. Diagnostic challenges can also be further magnified in patients co-infected with HIV and TB. In addition to these drawbacks, none of these techniques provide quantitative results that can be used to monitor treatment effects.

Diagnostic methods that do not rely on sputum exist but do not overcome many of the problems described above. For example, non-sputum-based Interferon-γ release assays (IGRAs), which measure ex vivo immune responses in patient blood samples after the addition of Mtb virulence antigens are ineffective at distinguishing between active TB and remote latent TB infection (LTBI) and perform poorly in diagnosing patients with EPTB or with HIV/TB co-infections. Methods for direct detection of Mtb virulence antigens circulating in the blood suffer from low sensitivity and specificity, likely due to epitope-masking effects of host proteins and homology with related antigens of several non-tuberculosis mycobacteria (NTM).

Thus, no method for rapid, accurate, and sensitive detection of active Mtb infections currently exists that is useful for all TB manifestations and patient populations, and TB continues to be associated with high morbidity and mortality.

Moreover, additional Mycobacterium species, generally referred to as nontuberculous mycobacteria (NTM) are also responsible for an increasing occurrence of debilitating pulmonary disease. However, unlike Mtb (notwithstanding the deficiencies thereof), there are currently no commercially available, FDA-approved molecular assays for diagnosis of NTM infections from clinical samples. Moreover, as there are a large number of NTM species, multiple assays would be required to cover the spectrum of clinically relevant NTM. While there have been some laboratory-developed methods for one or multiple NTM species, these methods are not standardized thereby limiting their broader use. As a result there are no satisfactory methods available for rapid and accurate NTM diagnosis.

As described above, infectious disease agents extend beyond the genus Mycobacterium, including Ebola, Zika, HIV/AIDS and some that have yet to be discovered or evolved. These agents may spontaneously cause disease outbreaks and lead to global epidemics. Accordingly, there is a need for a rapid response platform that can address a new epidemic and be deployed quickly and aid in the treatment and containment.

### SUMMARY OF THE INVENTION

The invention provides compositions and methods that allow rapid, specific, and high-sensitivity quantification of infections in several classes of patients. The compositions include energy-mediating nanoparticles (ENPs) that facilitate transfer of laser energy to enhance the sensitivity of a mass spectrometer for target peptides. The ENPs can be any type of nanoparticle, can include magnetic or non-magnetic material, and include a material, such as silica, which allows the nanoparticles to act as co-matrices for matrix-assisted laser desorption/ionization—time of flight mass spectrometry (MALDI-TOF MS). However, one skilled in the art will appreciate that the nanoparticles of the invention can be used with other mass spectrometry techniques as discussed below or with other assay types (e.g., immunoassay, biosensor, immunoPCR).

The ENPs may, optionally, also be conjugated to antibodies that bind to signature peptides of antigens. In one aspect of the present invention, the antibodies may be non-naturally-occurring antibodies specifically developed to selectively bind to these signature peptides. By way of example, certain signature peptides may be secreted during infection, such as CFP-10, ESAT-6, MPB64, Ag85B, and LAM for TB or VP40 for Ebola, etc. Thus, the nanoparticles can be used to capture disease-specific peptides from a patient sample, such as a blood-based sample, sputum, or other bodily fluid, and quantify the levels of the peptides, allowing the level of infection to be determined.

In one example, and without limitation thereto, the ENPs may be conjugated to antibodies that bind to signature peptides of Mtb antigens by including one or more of an antibody that binds specifically to a peptide of ESAT-6, and an antibody that binds specifically to a peptide of CFP-10. The antibodies may be conjugated to the surface of the ENP or to one or more internal surfaces of the pores of the ENPs. Specific, non-limiting examples may include an antibody that binds to a peptide of ESAT-6 derived from a polypeptide having the amino acid sequence of SEQ ID NO: 1, and more specifically to a peptide that has an amino acid sequence of any one or more of SEQ ID NOS: 1-2. Additionally or alternatively, an antibody may bind to a peptide of CFP-10 derived from a polypeptide having the amino acid sequence of SEQ ID NO: 3, and more specifically to a peptide that has an amino acid sequence of any one or more of SEQ ID NOS: 3-7.

In an aspect of the present invention, the nanoparticles may have any shape, such as but not limited to disks, rods, sphere, cylinders and the like. The nanoparticles may be solid or porous. In certain embodiments, the nanoparticles are disk-shaped and have pores that run approximately parallel to the axis of the disk. The nanoparticles may also include a material that enhances MALDI mass spectrometry signal by increasing peptide desorption/ionization. The material that enhances mass spectrometry signal may be a coating on the particle's outer surface, or in certain embodiments on an internal surface of the pores of the nanoparticle. For example, the outer and/or internal surface of the nanoparticle may be coated with silica, gold, a metal oxide or a metal oxide-hydroxide.

In accordance with an aspect of the present invention, the nanoparticles may be configured to absorb ultraviolet (UV) and/or visible light. For example, the nanoparticles may absorb light having a wavelength of about 100 to about 500 nm. The nanoparticles may have an absorbance peak of about 300 nm, about 325 nm, about 350 nm, 375 nm, about 400 nm, about 425 nm, 450 nm, about 475 nm, or about 500 nm.

Moreover, the antibodies provided herein may be directed against specific peptides within a specific antigen domain, thereby distinguishing one specific infectious species from other similar species, such as those within the same genus. In one non-limiting example, the antibodies may be directed against specific peptides resulting from digestion of Mtb CFP-10 and ESAT-6. In this manner, the compositions and methods of the present invention may distinguish between Mtb and NTM infections and are not hampered by epitope-masking effects of host proteins.

The invention also provides methods of determining the level of infection in a patient. The methods may include isolating signature peptides of pathogen-derived or disease associated antigens from patient samples, such as blood-based samples (whole blood, plasma, or serum), sputum, or other bodily fluids. The subject may be infected with more than one infectious agent. For example, the subject may be infected Mtb and with a virus, such as HIV. Isolation of the peptides may entail treating the sample in a manner that generates peptides of one or more target proteins indicative of the infection responsible for the suspected disease, contacting the sample with nanoparticles of the invention, and removing unbound peptides from the nanoparticles. The treating and contacting steps may be performed sequentially or simultaneously. The sample may be treated in any manner that breaks down proteins or polypeptides into peptides. For example, the sample may be digested with a protease, such as trypsin. In one aspect of the present invention, the sample is treated with trypsin while being exposed to microwave radiation.

The methods also include analyzing peptides in the sample by mass spectrometry and quantifying the levels of the signature peptides, thereby allowing the level of infection to be determined. The methods may entail contacting a sample with an ENP that is conjugated to include one or more antibodies selected to bind the isolated signature peptide(s). The ENP may also contain material that acts as a co-matrix for MALDI. In accordance with an aspect of the present invention, the peptides may be analyzed by matrix-assisted laser desorption/ionization-time of flight mass spectrometry (MALDI-TOF MS), thereby taking advantage of the co-matrix composition of the nanoparticles. However, one skilled in the art will recognize that other mass spectrometry approaches may be used to analyze the peptides. Other exemplary mass spectrometry techniques for peptide analysis that are suitable with methods of the invention include electrospray ionization, fast atom bombardment, chemical ionization, electron impact ionization, or atmospheric-pressure chemical ionization. Mass spectrometry may be performed while the peptides are bound to the nanoparticle. Alternatively, the peptides may be eluted from the nanoparticle, and mass spectrometry may be performed on the eluted peptides.

The methods of the present invention may be used to detect the expression of pathogen-derived proteins and virulence factors to distinguish latent infections and active disease. The sensitivity and specificity afforded by the present nanoparticles allows for reliable detection of an infection in all types of patients, including, for example, adult and pediatric patients, HIV-positive and HIV-negative patients, etc. In addition, because the methods permit pathogen levels to be quantified, they are useful for evaluating the severity of infections and for monitoring the response of an infection to treatment. Moreover, the nanoparticles of the present invention maybe used to distinguish disease levels, such as but not limited to latent, transitioning, active and early and late stage disease.

The compositions and methods described herein are useful in diagnostic applications because they overcome several of the limitations of existing approaches for assessing infection. For example, because the compositions of the present invention are effective with blood-based patient samples, they are useful in diagnosing patients with EPTB or who otherwise have low bacterial loads in their sputum.

### BRIEF DESCRIPTION OF THE DRAWINGS

FIG. 1 shows a schematic illustration of different types of ENPs which may undergo particle modification;

FIG. 2A shows a schematic of sample processing where an affinity molecule against a host protein is used to enrich a target protein;

FIG. 2B shows a schematic of sample processing where an affinity molecule against a target peptide that may be enriched from a complex patient sample;

FIG. 3 shows a general flowchart of an exemplary method in accordance with the present invention;

FIG. 4 shows a flow chart for peptide biomarker selection for a selected pathogen target;

FIG. 5 is a table of Mycobacteria species including SEQ ID NOS: 9-29 where bold underline text depicts amino acids that differ among two target peptide and asterisks (*) shown below the peptide alignment indicate complete sequence identify at an amino acid position and underlined asterisks indicate conserved amino acid sequences that can be recognized by a capture antibody or other affinity molecule;

FIG. 6 is a table of exemplary candidate biomarker peptides for differentiation of the known ebolavirus species generated by trypsin digestion of the ebolavirus matrix protein VP40 including SEQ ID NOS: 30-34 where bold underline text depicts amino acids that differ with a target peptide region conserved among all ebolavirus species and asterisks (*) shown below the peptide alignment indicate complete sequence identify at an amino acid position and underlined asterisks indicate conserved amino acid sequences that can be recognized by a capture antibody or other affinity molecule;

FIG. 7 shows an exemplary target peptide suitable for analysis with a panel of species-specific peptide antibodies where the peptide alignment depicts a region shared among a virulence factor conserved among multiple related pathogen species and where colored circles indicate amino acid variants at a given position;

FIG. 8 shows an exemplary pathogen-derived peptide biomarker suitable for multiplex analysis with a single peptide antibody where the peptide alignment depicts a region shared among a virulence factor conserved among multiple related pathogen species and where colored circles indicate amino acid variants at a given position;

FIG. 9 shows a schematic illustration of an exemplary energy-mediating nanoparticle (ENP) platform in accordance with an aspect of the present invention;

FIG. 10A illustrates method optimization and multiplex quantification of Mtb target peptides with MS signal intensity of target peptides analyzed alone (no ENPs) or with graphene, silver (Au) gold (Au), silicon (Si), silica nanoparticles (ENPs), or NanoDisks;

FIG. 10B shows a scanning electron microscope image of NanoDisk structure;

FIG. 10C shows a transmission electron microscope image of cross-sectional structure;

FIG. 10D shows silica modification of NanoDisk inner pore surfaces;

FIG. 10E shows intensity in an antigen-spiked healthy serum sample that was trypsin digested overnight (12 hr) or by rapid microwave-irradiation (20 min) and then analyzed without immunoprecipitation (IP), after IP and elution from target-specific Dynabeads or NanoDisks, or by NanoDisk-MS (CFP-10 1593.75 (SEQ ID NO: 56—left) and ESAT-6 1900.95 (SEQ ID NO: 35—right));

FIG. 10F shows calibration curves for CFP-10 and ESAT-6 quantitation in serum (n=3; R2>0.98)];

FIG. 10G shows MS spectra of CFP-10 and ESAT-6 peptides (m/z 1593.75 (SEQ ID NO: 56) and 1900.95 (SEQ ID NO: 35)) and their internal standards (m/z 1603.60 and 1910.80) in sera of a healthy control, and a TB case analyzed by NanoDisk-MS;

FIG. 10H shows MS spectra of CFP-10 and ESAT-6 peptides (m/z 1593.75 (SEQ ID NO: 56) and 1900.95 (SEQ ID NO: 35)) and their internal standards (m/z 1603.60 and 1910.80) in serum of a TB case analyzed by MALDI-TOF/TOF MS with or without Dynabead enrichment or by NanoDisk-MS after 32-fold serum dilution (mean±SEM; n=3; ***p<0.001);

FIG. 11 shows identification of active TB in pediatric and adult patients using serum CFP-10 and ESAT-6 concentrations in adult HIV-negative and HIV-positive groups, where each column represents a patient sample, ranked by CFP-10 concentration, for which antigen levels are shown by color intensity in the matching gradient bars;

FIG. 12A shows CFP-10 and ESAT-6 quantitation in archived serum samples of HIV−/TB+;

FIG. 12B shows CFP-10 and ESAT-6 quantitation in archived serum samples of HIV+/TB+ patients during anti-TB therapy (2-11 vs. 8-21 month);

FIG. 12C shows CFP-10 and ESAT-6 quantitation in serum samples of prospectively enrolled patients with active TB collected prior to treatment and 4 or 9 days after treatment initiation;

FIG. 13 shows MALDI-TOF MS signals of tryptic digested CFP-10 and ESAT-6 fragments;

FIG. 14 shows LC-MS/MS spectra of target peptides from recombinant CFP-10 and ESAT-6;

FIG. 15 shows a sequence alignment of the Mtb CFP-10 and ESAT-6 target peptide regions;

FIG. 16 shows MALDI-TOF MS spectra of healthy serum spiked with CFP-10 and ESAT-6 after traditional overnight or microwave assisted trypsin digestion and desalting;

FIG. 17 shows UV-vis NanoDisk spectra;

FIG. 18 shows scanning and transmission electron microscopy of oxidized NanoDisks;

FIG. 19 shows MALDI-TOF MS signal enhancement within MALDI-TOF MS spectra of CFP-10 1593.75- and ESAT-6 1900.95 generated with and without NanoDisks as a MALDI co-matrix;

FIG. 20 is a schematic illustration of the NanoDisk surface functionalization method;

FIG. 21 shows representative MS spectra of CFP-10 and ESAT-6 peptides (m/z 1593.75 and 1900.95) and their respective internal standards in: 2× diluted patient serum detected by MALDI-TOF/TOF MS without enrichment (upper); 2× diluted patient serum detected by MALDI-TOF/TOF MS with Dynabeads enrichment (middle); 2× diluted patient serum detected by NanoDisk-MS method; and

FIG. 22 shows receiving operating characteristic (ROC) curves for (A) CFP-10 and (B) ESAT-6 using active TB cases as patients and non-TB subjects as controls.

### DETAILED DESCRIPTION

The present invention generally relates to compositions and method of use of such compositions for determining a level of a specific disease-associated biomarker in a subject and to assess disease severity in a subject. As used throughout the Specification and Claims, the term “disease” shall encompass any stage of infection or disease level, such as but not limited to latent, active, early stage, or late stage disease or transitioning from latent to active disease, unless otherwise specified or indicated by context. Further, as used throughout the Specification and Claims, the term “infection” shall encompass any pathogen level independent of its association with active, early stage, or late stage disease or the transition from latent to active disease. The compositions include energy-mediating nanoparticles (ENPs) that optionally have affinity ligands, such as antibodies, conjugated thereto. As discussed above, the ENPs may be conjugated to one or more antibodies that bind to signature peptide(s) of antigens that are secreted during infection, such as CFP-10, ESAT-6, MPB64, Ag85B, and LAM for TB, or VP40 for Ebola, etc. The ENPs may also include a material, such as silica, that allows the nanoparticles to act as a co-matrix for matrix-assisted laser desorption/ionization-time of flight mass spectrometry (MALDI-TOF MS). Thus, the ENPs can be used to capture disease-specific peptides from a patient sample (e.g., blood-based patient sample) and quantify the levels of these peptides. In one aspect of the present invention, quantification of disease-specific peptides may allow the level/severity of disease to be determined and subsequently monitored during treatment.

In accordance with an aspect of the invention, methods of determining the level of disease in a patient are provided. An exemplary method may include isolating signature peptide(s) of one or more diseases from patient samples (e.g., blood-based patient samples) by, for example, digesting the sample with a protease or chemical cleavage agent with or without exposing the sample to microwave radiation. The digested proteins may be thereafter exposed to ENPs modified to include affinity ligands complementary to the signature peptide(s) so as to capture and enrich the signature peptide(s) for further analysis. Additionally or alternatively, the exemplary method may also include isolating whole proteins from patient samples using ENPs in accordance with the present invention. Target proteins may bind to the ENPs such that all or a substantial portion of the remaining milieu may be removed so as to yield the isolated target proteins. The isolated proteins may then undergo digestion as described above, with the resultant peptides being captured by a second set a nanoparticles which include relevant affinity ligands.

In accordance with a further aspect of the present invention, the exemplary method may also include analyzing peptide(s) in the sample by mass spectrometry and quantifying the levels of the signature peptides, which may allow the level of disease to be determined. In particular embodiments, the exemplary method of the invention may be carried out using the ENPs described herein, with the analysis being performed by MALDI-TOF MS directly on the nanoparticles so as to ionize the captured peptides. The ENPs may enhance the ionization of captured peptides, resulting in increased sensitivity of detection in MALDI-TOF mass spectrometry.

### Energy-mediating Nanoparticles (ENPs)

In the last decade, desorption/ionization mass spectrometry (DIMS) has gained much attention in clinical diagnostic applications because of its high accuracy and high throughput screening capabilities. However, the use of chemical matrices introduces certain limitations including the need to select matrices compatible with the target analytes, the interference of chemical matrices in the low-molecular weight range, and poor quality of data (i.e., low sensitivity and poor reproducibility). To overcome these issues, nanostructured materials such as silicon (Si) nanowires and porous Si substrates have been developed to replace chemical matrices. The high-energy laser absorptions on the nanostructured Si materials strongly enhance the efficiency of analyte desorption and ionization. Nevertheless, one common problem in using Si substrates is their high affinity for hydrocarbons and other ambient species. Desorption of these species generates background interference in the low end of the mass spectrum. A second problem is related to the nature of the ion isolation mechanism. Many biomolecules easily detectable by Electrospray Ionization MS and Nuclear Magnetic Resonance are not amenable to detection by DIMS because the latter method has a strong selectivity toward compounds that have a high-proton affinity, low-ionization energy, and high-stabilization energy. A third problem with DIMS is poor quantitative performance due to the poor spot-to-spot precision when depositing samples on the target plate.

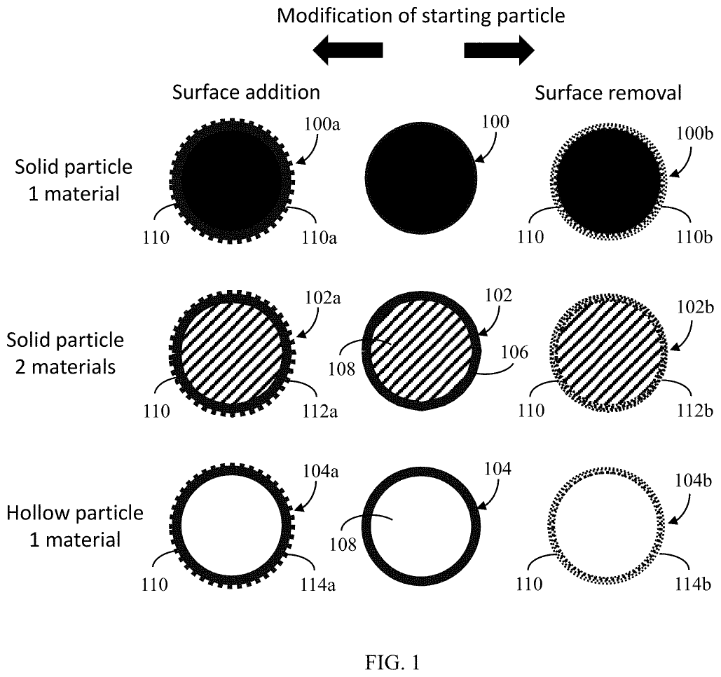

Turning now to the figures, and with particular reference to FIG. 1, to address the obstacles posed by current DIMS methods for detecting low abundance biomarkers, exemplary energy-mediating nanoparticles (ENPs) are shown. ENPs may provide for enhanced laser desorption/ionization of analytes as will be described in greater detail below. “Nanoparticles” refer to particles that have dimensions typically measured on a nanometer scale, e.g., 1-1000 nm. “Microparticles” refer to particles that have dimensions typically measured on a micrometer scale, e.g., 1-1000 μm. However, the boundary between “nanoparticles” and “microparticles” is not discrete, and the terms may be used interchangeably herein for particles having dimensions high on the nanometer scale or low on the micrometer scale, for example, particles having diameters from 100 nm (0.1 μm) to about 10,000 nm (10 μm). By controlling the size, surface density, and distribution of deposited materials on the ENPs, the optical and surface properties of the ENPs can be selectively regulated, thereby permitting controllable generation of appropriate ENPs for very sensitive detection of a variety of target peptides.

In accordance with an aspect of the present invention, ENPs may have any shape and size suitable for the application. For example and without limitation thereto, ENPs may be formed into any shape, such as a disk, rod, sphere, cylinder or any other suitable configuration. For instance, ENPs may be shaped like a sphere having a diameter of about 500 nm, about 600 nm, about 700 nm, about 800 nm, about 900 nm, about 1000 nm, about 1100 nm, about 1200 nm, about 1300 nm, about 1400 nm, about 1500 nm, from about 200 nm to about 2000 nm, from about 500 nm to about 2000 nm, from about 800 nm to about 2000 nm, from about 200 nm to about 1500 nm, from about 500 nm to about 1500 nm, or from about 800 nm to about 1500 nm.

In a further example, ENPs may be shaped like a disk and have diameters similar to those of the above spheres and a height of about 100 nm, about 200 nm, about 300 nm, about 400 nm, about 500 nm, about 600 nm, about 700 nm, about 800 nm, about 900 nm, about 1000 nm, from about 100 nm to about 1000 nm, from about 200 nm to about 1000 nm, from about 400 nm to about 1000 nm, from about 100 nm to about 500 nm, from about 200 nm to about 500 nm, or from about 300 nm to about 500 nm.

As shown in FIG. 1, ENPs may be formed as a solid nanoparticle 100, 102 or as a hollow nanoparticle 104. In accordance with an aspect of the present invention, ENPS 100, 102, 104 may include materials that allow the nanoparticle to act as a co-matrix for matrix-assisted laser desorption/ionization-time of flight mass spectrometry (MALDI-TOF MS). Preferably, the ENP material functions as a co-matrix to enhance mass spectrometry signal by improving peptide desorption/ionization efficiency. For example, solid ENP 100 and hollow ENP 104 may each be formed of one material, such as silica, gold, a metal oxide or a metal oxide-hydroxide which enhances mass spectrometry signal. Alternatively, the material that enhances mass spectrometry signal (silica or gold, etc.) may be formed as a coated layer 106 on the surface of ENP 102. In this instance, the core 108 of ENP 102 may be a second material, such as silicon, or other material that can function as a co-matrix. Core 108 may also be comprised of a magnetic material, such as but not limited to iron, nickel or cobalt and their oxides and alloys thereof. The magnetic core may aid in separating ENP 102 from the sample milieu, as will be discussed in greater detail below. In accordance with a further aspect of the present invention, any or all of ENPS 100, 102, 104 may also undergo surface modification, such as surface addition 100a, 102a, 104a or surface removal 100b, 102b, 104b. Hereinafter, reference to ENP 100 shall include ENPs 100, 100a, 100b; reference to ENP 102 shall include ENPs 102, 102a, 102b; and reference to ENP 104 shall include ENPs 104, 104a, 104b unless otherwise indicated or as dictated by context. These surface modifications may produce respective textured surfaces 110a, 110b; 112a, 112b; and 114a, 114b, (collectively referred to herein as textured surface 110), defining features such as pores, spikes, ridges and valleys or other similar morphologies (collectively referred to herein as pores). Textured surface 110 may be formed of co-matrix material or may be formed of a non-co-matrix material which is then coated with co-matrix material. In another aspect of the invention, ENPs 100, 102 (core 108 and/or coated layer 106), 104 may also be magnetic or have magnetic properties.

The optional pores defined by textured surface 110 may have any shape. By way of example and without limitation thereto, the pores may have an average maximum diameter of about 10 nm, about 20 nm, about 30 nm, about 40 nm, about 50 nm, about 60 nm, about 70 nm, about 80 nm, about 90 nm, about 100 nm. The pores may have average diameters from about 5 nm to about 200 nm, from about 10 nm to about 100 nm, from about 20 nm to about 80 nm, from about 30 nm to about 60 nm.

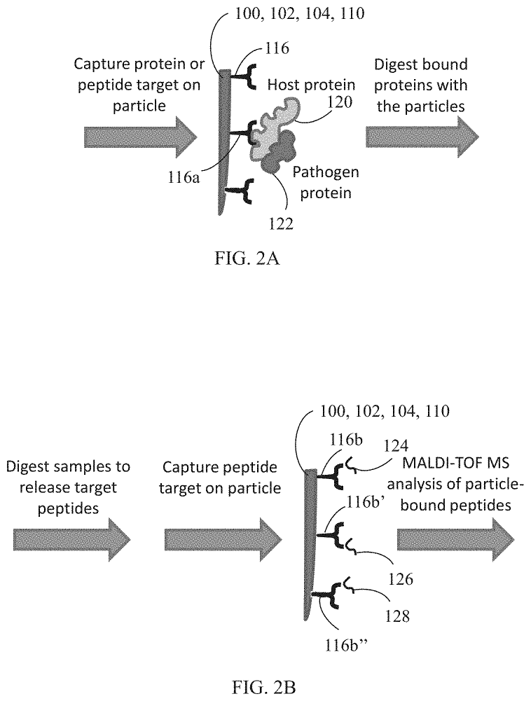

With reference to FIGS. 2A and 2B, all or a portion of the outer surface of ENP 100, 102, 104 or textured surface 110 may be modified to include one or more affinity agents, such as but not limited to an antibody 116. By way of example, as shown in FIG. 2A, antibody 116 may comprise an antibody 116a with high affinity for a specific host protein 120 such that host protein 120 binds with antibody 116a. Some potential pathogen biomarkers significantly interact with host proteins and this can be employed to enrich proteins that may lack peptides containing conserved regions suitable to their direct isolation. Target pathogen protein 122 may have a high affinity for host protein 120 whereby target pathogen protein 122 selectively binds to host protein 120. In this manner, the remaining sample milieu may be removed as described above such that host protein 120 and bound target pathogen protein 122 are substantially isolated and enriched. Target pathogen protein 122 may then undergo proteolysis as will be described in greater detail below.

As shown in FIG. 2B, a peptide digestion may be exposed to ENP 100, 102, 104 such that target peptides 124 indicative of an infection may bind to an antibody 116b. The protein digestion may be derived from the isolated target pathogen protein 122 enriched in FIG. 2A, or may be derived from a digestion of an entire protein fraction of a patient sample or a portion thereof. In this manner, the affinity agents may selectively and specifically bind to a pathogen-specific peptide, thereby increasing peptide enrichment such that subsequent detection techniques may detect and identify pathogen-specific peptides in the femtomolar concentration range.

By way of example and without limitation thereto, antibody 116b′ may bind specifically to a peptide 126 of ESAT-6 and/or antibody 116b″ may bind specifically to a peptide 128 of CFP-10, wherein ESAT-6 and CFP-10 are each biomolecules indicative of Mtb. In this example, antibody 116b′ may bind to a peptide of ESAT-6 that has an amino acid sequence of either or both of SEQ ID NOS: 1-2. Exemplary antibody 116b″ may bind to a peptide of CFP-10 that has an amino acid sequence of SEQ ID NOS: 3-7. The term “sequence,” when referring to amino acids, relates to all or a portion of the linear N-terminal to C-terminal order of amino acids within a given amino acid chain, e.g., a peptide or polypeptide.

Antibodies 116 may be conjugated to a nanoparticle, such as ENP 100, 102, 104 or textured surface 110, by any method known in the art. By way of example, antibodies 116 may be irreversibly conjugated to ENP 100, 102, 104 or textured surface 110. For example, and without limitation thereto, silica-coated surfaces of the ENPs may be treated with (3-Glycidyloxypropyl)trimethoxysilane (GLYMO, Sigma-Aldrich), a bifunctional organosilane possessing a reactive organic epoxide and hydrolyzable inorganic methoxysilyl group. The methoxysilyl group reacts with the silica surface of the nanoparticle, and the organic epoxide forms a covalent bond with antibody 116. Alternatively, antibody 116 may be reversibly conjugated to ENP 100, 102, 104 or textured surface 110, by for example, reversible addition—fragmentation chain transfer (RAFT) polymerization; adding a metal-chelating histidine tag to antibody 116 and covalently attaching another chelating agent, e.g., nitriloacetic acid to the ENP; or using polyadenylate-streptavidin linkers to link biotinylated antibodies 116 to ENP 100, 102, 104 or textured surface 110 which have been coated with polythymine oligonucleotides.

In accordance with an aspect of the present invention, ENP 100, 102, 104 may be configured to absorb ultraviolet (UV) and/or visible light. For example, ENP 100, 102, 104 may be configured to absorb light having a wavelength of about 300 to about 450 nm. ENP 100, 102, 104 may have an absorbance peak of about 300 nm, about 325 nm, about 350 nm, 375 nm, about 400 nm, about 425 nm, 450 nm, about 475 nm, or about 500 nm. As a result, ENP 100, 102, 104 may enhance desorption/ionization efficiency of peptides when performing MALDI-TOF MS. However, it should be noted the ENP 100, 102, 104 may also be used in variety of detection methods, such as, for example, other types of mass spectrometry or non-mass spectrometric analytical methods such as immunoassays and immune-PCR, or as components in biosensor systems that rely on chemical, electrical, electrochemical and/or optical signals for detection and/or analysis.

### Detection of Infections in Patient Samples

Various embodiments described herein relate to the detection of one or more disease-specific biomolecules in a sample. The sample can comprise, or be derived from, at least one biological fluid selected from the group consisting of blood serum, blood plasma, blood, urine, seminal fluid, seminal plasma, pleural fluid, ascites, nipple aspirate, feces, and saliva. In particular, the sample may contain, or be derived from, at least one bodily fluid, including, without limitation, sputum, pleural effusion, cerebrospinal fluids, urine, whole blood, serum, and plasma. Preferably the bodily fluid will be obtained from a vertebrate mammal, and in particular, a human having, suspected of having, and/or at risk for developing, an infection.

As used herein, the terms “patient”, “host”, or “subject” refer to any entity that can receive one or more of the pharmaceutical compositions disclosed herein. Preferably, the subject is a vertebrate animal, which is intended to denote any animal species (and preferably, a mammalian species, such as a human being). In certain embodiments, a “patient” refers to any animal host including without limitation any mammalian host. Preferably, the term refers to any mammalian host, the latter including but not limited to, human and non-human primates, bovines, canines, caprines, cavines, corvines, epines, equines, felines, hircines, lapines, leporines, lupines, murines, ovines, porcines, ranines, racines, vulpines, and the like, including livestock, zoological specimens, exotics, as well as companion animals, pets, and any animal under the care of a veterinary practitioner. A patient can be of any age at which the patient is able to respond to inoculation by generating an immune response.

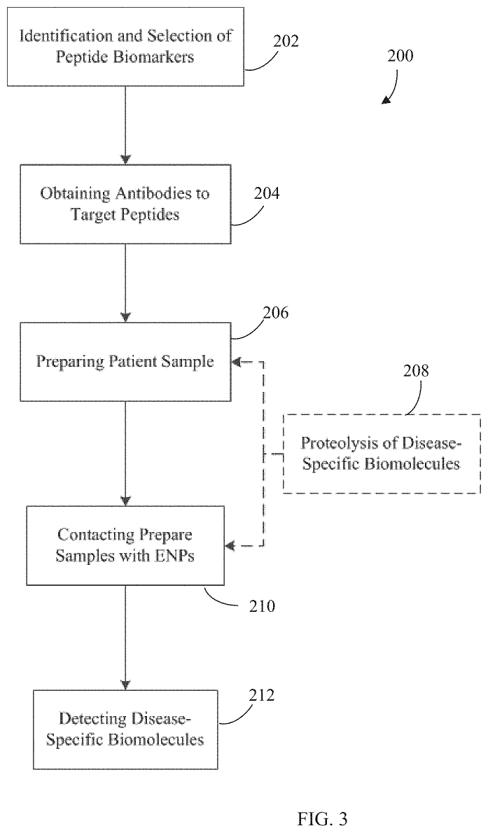

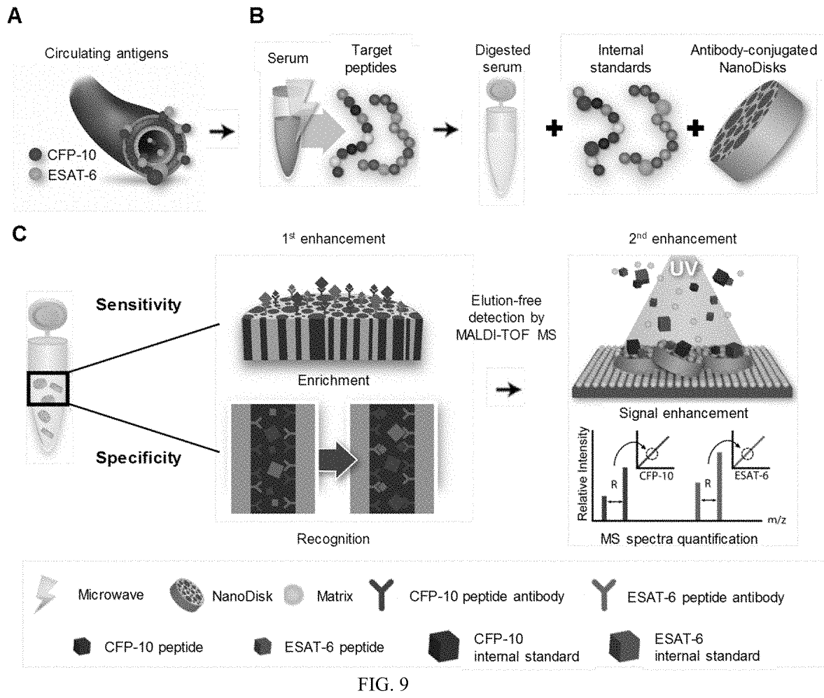

As will be discussed in greater detail below, there are many disease-specific biomarkers suitable for detection by the methods described herein, including, but not limited to, biological molecules such as proteins, peptides, polypeptides, antibodies, polynucleotides, oligonucleotides, nucleic acids, and/or polysaccharides. In accordance with an aspect of the present invention, an exemplary method 200 for detecting of one or more disease-specific biomolecules in a sample is shown in FIG. 3, with additional reference to FIG. 9. FIG. 9 shows a schematic illustration of an exemplary energy-mediating nanoparticle (ENP) platform in accordance with an aspect of the present invention. The exemplary ENP platform may be arranged as a NanoDisk, although those skilled in the art should understand that alternative platforms may be used, such as but not limited to rods, sphere, cylinders and the like. (A) CFP-10 and ESAT-6 are secreted into the circulation from active Mtb infections. (B) Serum samples are subjected to microwave-assisted tryptic digestion and mixed with functionalized NanoDisks and stable isotope-labeled internal standard peptides. (C) Peptide quantification. Step 1: Recognition and enrichment of target peptides and stable isotope-labeled internal standard peptides by antibody-conjugated NanoDisks. Step 2: A NanoDisk effect to enhance MALDI signal allows quantification of target peptide at low concentrations, determined by MS intensity ratio of target and isotope-labeled internal standard peptides. Each of the respective steps of method 200 will be described in greater detail in the paragraphs below.

### Identifying and Selecting Peptide Biomarkers

At step 202 of exemplary method 200, one or more peptides are selected wherein the peptides are species-specific such that detection of a peptide indicates the presence of a specific species. In accordance with an aspect of the present invention, protein and/or peptide sequences may be selected through use of a database, such as the publically available online UniProt database, www.uniprot.org, or other similar database repository.

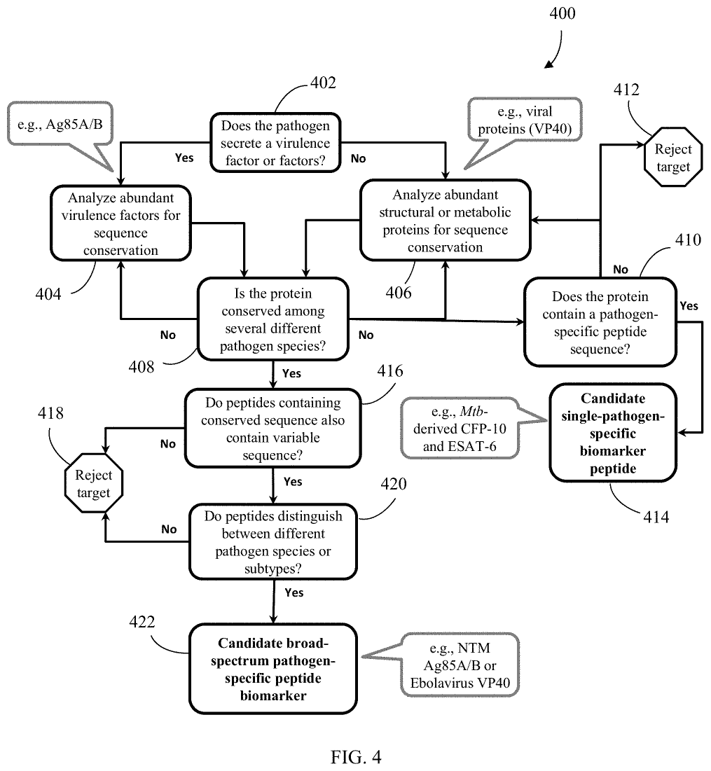

Alternatively, as shown in FIG. 4, a general algorithm 400 may be employed to isolate and identify a species-specific peptide. At step 402, it must first be determined if the pathogen (e.g., M. tuberculosis, Ebola, HIV/AIDS) secretes a virulence factor or factors. If one or more virulence factors are secreted, it is next determined if there is sequence conservation (step 404) among the factors. If one or more virulence factors are not secreted, the pathogen's structural and metabolic proteins are analyzed for sequence conservation (step 406). In either event, it is next determined whether the virulence factors or pathogen proteins are conserved among different pathogen species (step 408).

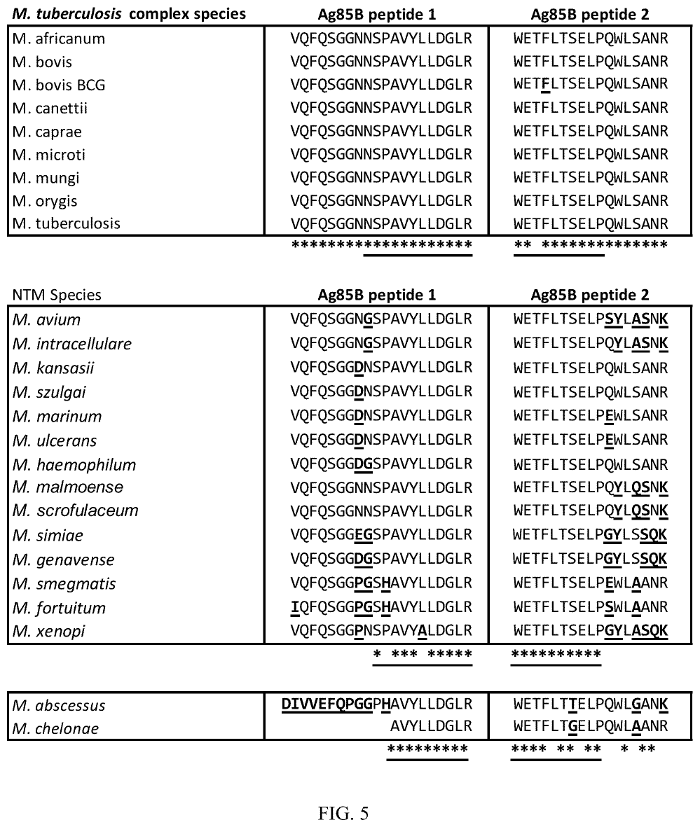

If the protein sequence is not conserved, a next virulence factor (if available) is analyzed (step 404) or a next pathogen protein is analyzed (step 406). Additionally or alternatively, the protein may be analyzed to determine if the protein contains a pathogen-specific peptide sequence (step 410). If no, the protein target is rejected (step 412) or, if yes, the peptide sequence is accepted as a candidate single-pathogen-specific biomarker peptide (step 414); see, for example, FIG. 5 regarding genus Mycobacterium and two peptides of protein Ag85B; ESAT-6 and CFP-10 examples in SEQ. ID NOS. 1-7. Target-specific ENPs may then be fabricated for each individually identified single-pathogen-specific biomarker peptide. See, e.g., FIG. 7A.

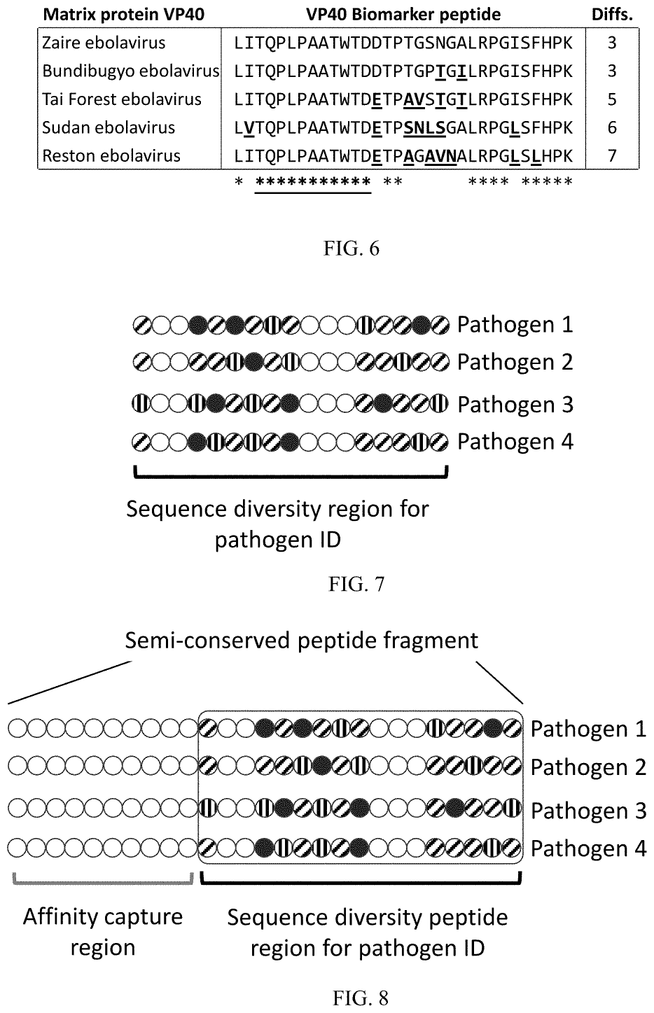

If the protein is conserved among several different pathogen species, the protein is analyzed to determine if the conserved sequence also includes a variable sequence (step 416). If there is no variable sequence, the protein is rejected at step 418 due to lack of protein specificity. That is, detecting of the protein will not lead to positive identification of a single pathogen. However, if there is at least one variable sequence, it is next determined whether the variance distinguishes between pathogen species or subtypes (step 420). Again, if no, the protein is rejected (step 418). If the variable sequence does distinguish between species or subtypes, that protein is selected at step 422 as a candidate broad-spectrum pathogen-specific peptide biomarker having an affinity capture region; see, for example, FIGS. 6 and 8 regarding a portion of protein VP40 for various ebolavirus species and SEQ. ID NO. 8 identifying a suitable affinity capture region peptide sequence for antibody targeting.

As shown in FIGS. 5 and 6, bold underline text within the peptide sequence indicates amino acids that differ among two target peptides. Asterisks at the bottom of the list indicate complete sequence identity (sequence conservation) while underlined asterisks indicate a conserved sequence that can be recognized by an affinity agent, such as an antibody 116.

### Obtaining Antibodies to Target Peptides

After identifying and selecting target peptides, antibodies to these peptides may be obtained at step 204. Antibodies may be fabricated using techniques known in the art and may be manufactured on-site or purchased from a suitable vendor. To ensure selectivity and specificity, an antibody is configured to selectively bind to the target peptide or an affinity capture region shared by peptides of related species as will described in greater detail below. Without being held to any particular theory, antibodies may bind specifically to peptides with higher affinity to the antigenic peptide than to other peptides of similar size, similar amino acid composition, or similar but not identical amino acid sequence, e.g., peptides having one or more amino acid substitutions.

For example, an antibody may have a Kd (the equilibrium dissociation constant between an antibody and its antigen) for the antigenic peptide that is about 100-fold, 1000-fold, 10,000-fold or more lower than its Kd for a non-antigenic peptide. As described above, for the exemplary ESAT-6 and CFP-10 antigens indicative of an Mtb infection, the antibodies to ESAT-6 may bind to a peptide having an amino acid sequence of any of SEQ ID NOS: 1-2 while the antibodies to CFP-10 may bind to a peptide having an amino acid sequence of any of SEQ ID NOS: 3-7. In a further example, detecting peptides of multiple ebolavirus species may include an antibody selected to target the affinity target region of SEQ ID NO: 8. MALDI-TOF analysis may then differentiate each ebolavirus species from the other, as will be described in greater detail below.

It should be understood by one skilled in the art that mammals naturally generate antibodies to foreign molecules and that antibodies to the peptides may exist in nature. The antibodies of the invention are considered “non-naturally-occurring” if they are not known to exist in nature and are produced by human intervention. Thus, antibodies are considered non-naturally-occurring within the scope of the invention if they result from exposure of an animal to an antigen for the purpose of producing antibodies to that antigen, even if the production of the antibody is a normal immune response of the animal.

### Preparing Patient Sample

Once one or more biomarkers have been identified and selected, and antibodies selective from those biomarkers have been obtained, patient samples may then be prepared for analysis in step 206. The patient sample may be any type of fluid obtained from a patient. For example and without limitation, the patient sample may be sputum, pleural effusion, cerebrospinal fluid, urine, serum, plasma, whole blood, tissue filtrate, tissue lysate, tissue homogenate, or lymph fluid. Preferably, the patient sample is a blood-based sample. The blood-based sample may be any material derived from blood, such as whole blood, plasma, or serum. The blood can be obtained by standard phlebotomy procedures and then separated.

Typical separation methods for preparing a plasma sample include centrifugation of the blood sample. For example, immediately following blood draw, protease inhibitors and/or anticoagulants can be added to the blood sample. The tube is then cooled and centrifuged, and can subsequently be placed on ice. The resultant sample is separated into the following components: a clear solution of blood plasma in the upper phase; the buffy coat, which is a thin layer of leukocytes mixed with platelets; and erythrocytes (red blood cells). Typically, 8.5 mL of whole blood will yield about 2.5-3.0 mL of plasma.

Blood serum is prepared in a very similar fashion. Venous blood is collected, followed by mixing of protease inhibitors and coagulant with the blood by inversion. The blood is allowed to clot by standing tubes vertically at room temperature. The blood is then centrifuged, wherein the resultant supernatant is the designated serum. The serum sample should subsequently be placed on ice.

In some embodiments, the sample suspected of comprising one or more disease-specific biomolecules may be “pre-concentrated” before application onto ENP 100, 102, 104. Disease-specific biomolecules, if present in the sample, can be pre-concentrated by one or more conventional methods, including for example, by precipitating the proteinaceous fraction of the sample. Standard protein precipitation methods are known to those of ordinary in the art and may include, for example, the use of ammonium sulfate. The resulting protein precipitate can then be separated from the aqueous fraction and dissolved in a suitable solvent or diluent before being applied onto ENP 100, 102, 104.

Alternatively, as described above with reference to FIG. 2A, the sample may be pre-concentrated by applying the sample onto ENP 100, 102, 104 which include affinity agents configured to selectively and specifically bind the target peptide(s). The sample may then undergo centrifugation whereby the ENP-bound protein or peptide may spin down to the bottom of the centrifuge tube. The remaining supernatant may be decanted and the ENP-bound peptide may then be dissolved in a suitable solvent or diluent.

In addition to centrifugation, samples may be further prepared through filtration. Various filtration media for removal of particles includes filer paper, such as cellulose and membrane filters using regenerated cellulose, cellulose acetate, nylon, PTFE, polypropylene, polyester, polyethersulfone, polycarbonate, and polyvinylpyrolidone. Various filtration media for removal of particulates and matrix interferences includes functionalized membranes, such as ion exchange membranes and affinity membranes, SPE (solid phase extraction) cartridges such as silica- and polymer-based cartridges; and SPE disks, such as PTFE- and fiberglass-based disks. Some of these filters can be provided in a disk format for loosely placing in filter holdings/housings while others are provided within a disposable tip that can be placed on, for example, standard blood collection tubes, while still others are provided in the form of an array with wells for receiving pipetted samples. Another type of filter includes spin filters. Spin filters consist of polypropylene centrifuge tubes with cellulose acetate filter membranes and are used in conjunction with centrifugation to remove particulates from samples, such as serum and plasma samples, typically diluted in aqueous buffers.

### Proteolysis of Disease-Specific Biomolecules

At optional step 208, the sample suspected of containing disease-specific biomolecules may be subjected to proteolysis prior to detection. The sample may be digested, for example, using one or more proteolytic agents, such as a protease or a peptidase, to proteolytically cleave one or more polypeptides or proteins present in the sample. Various protease or peptidase are known in the art, including, for example, serine proteases, cysteine proteases, aspartate proteases, threonine proteases, glutamic acid proteases, and metalloproteases. In accordance with an aspect of the invention, the protease may be trypsin, or an analog or an active fragment thereof. The proteolysis of the sample may be performed prior to analysis, or alternatively, upon or within the ENPs during analysis.

In some embodiments, the disease-specific biomolecule can be digested directly on or within the ENP itself (i.e., on-site proteolysis). The proteolytic products so produced can then be extracted from the ENP for further characterization or analysis. Extraction of the digestion products may be achieved by, for example, using one or more suitable elution buffers. In other embodiments, the biomolecules of interest, or the proteolytic byproducts thereof may be extracted from the ENP before being digested at a different site. The digestion product can comprise, for example, one or more different identifiable fragments of the disease-specific biomolecule.

By way of example and by no means limiting thereto, a M. tuberculosis-specific biomolecule is an ESAT-6 protein or peptide where the digestion product can comprise, for example, at least one peptide having a mass fingerprint at about 1895-1910 Da. The digestion product(s) can include, for example, at least one ESAT-6 fragment comprising, consisting essentially of, or alternatively, consisting of, the sequence of WDATATELNNALQNLAR (SEQ ID NO:1); at least one ESAT-6 fragment comprising, consisting essentially of, or alternatively, consisting of, the sequence of LAAAWGGSGSEAYQGVQQK (SEQ ID NO:2), or a combination of both fragments.

Continuing the above example, the M. tuberculosis-specific biomolecule is a CFP-10 protein or peptide where the digestion product can include, for example, at least one CFP-10 fragment resulting from proteolysis. In such embodiments, the digestion product(s) can include, for example, at least one CFP-10-specific peptide fragment that comprises, consists essentially of, or alternatively, consists of, the amino acid sequence of any one or more of:

(SEQ ID NO: 3)
TQIDQVESTAGSLQGQWR,
(SEQ ID NO: 4)
ADEEQQQALSSQMGF,
(SEQ ID NO: 5)
TDAATLAQEAGNFER,
(SEQ ID NO: 6)
GAAGTAAQAAVVR and
(SEQ ID NO: 7)
QAGVQYSR.

Other methods of proteolysis are possible within the scope of the invention. For example, proteolysis may be achieved or facilitated by exposing a sample to formic acid or other non-enzymatic proteolytic agent. Microwave radiation may be performed in addition to protease digestion. Samples may be proteolyzed by other methods, such as chemical breakage of peptide bonds, etc.

Contacting Prepared Samples with ENPs

In step 210, after a patient sample suspected of containing at least a first pathogen-specific biomarker antigen has been prepared for analysis (and optionally proteolyzed in step 208), the prepared sample is then contacted with a population of ENPs 100, 102, 104 that include one or more biomarker-specific antibodies 116 immobilized thereon under conditions to permit specific binding of a biomarker-specific antigen to one or more of the antibodies. The biomarker antigen-specific antibodies may include one or more specific antibodies, or one or more antigen-binding fragments thereof, or combinations thereof. As described above, ENPs 100, 102, 104 may also operate as co-matrices for enhanced MALDI desorption/ionization efficiency. Without being bound to any particular theory, it is contemplated that the ENPs may augment the vaporization and/or increase the ionization of one or more of the antibody-bound antigens during MALDI-TOF MS analysis, thereby increasing the sensitivity of the assay, and permitting detection of the target pathogen-specific biomarkers, even when present in the original patient samples in very low (i.e., nanomolar to femtomolar) concentrations.

### Detecting Disease-Specific Biomolecules

After contacting ENPs 100, 102, 104 in step 210, the presence of a disease-specific biomolecule may then be detected in step 212 using any one or more techniques known to those of ordinary skill in the art, including, without limitation, mass spectrometry, gel electrophoresis, chromatography, one or more bioassays, one or more immunological assays, or a combination of two or more such techniques.

In accordance with an aspect of the invention, mass spectrometry may be used to detect the presence of disease-specific proteins and peptides from a sample of interest. Any suitable ionization technique may be used, such as and without limitation thereto, atmospheric pressure ionization sources including matrix-assisted laser desorption/ionization (MALDI), surface-enhanced laser desorption/ionization (SELDI), electrospray ionization (ESI), nanoelectrospray ionization (nESI), desorption electrospray ionization (DESI), electrospray-assisted laser desorption/ionization (ELDI), direct analysis in real time (DART), and Atmospheric Pressure Dielectric Barrier Discharge Ionization (DBDI). Suitable mass analyzers may include time-of-flight (TOF), Fourier transform-ion cyclotron resonance (FT-ICR), quadrupole, or ion traps. In accordance with an aspect of the invention, the mass spectrometer may be coupled with another analytical modality, such as a liquid chromatograph (LC-MS) or capillary electrophoresis (CE-MS). In yet another aspect of the present invention, the mass spectrometer may be configured to conduct tandem mass spectrometry (MS-MS) or multistage mass spectrometry (MS″), and/or mass spectrometry. By way of example, MALDI-TOF MS may be employed to detect the presence of one or more disease-specific biomolecules whereby ENPs 100, 102, 104 may act as co-matrices with any suitable MALDI matrix for improved peptide desorption and ionization.

In accordance with an aspect of the present invention, the presence of a disease-specific biomolecule can be detected by, for example, finding one or more mass spectroscopy “fingerprints” unique to the particular biomolecule of interest. The mass fingerprints may be one or more enzymatic digestion products of the particular biomolecule of interest. Specific examples may be found in the below examples.

### Diagnostic Kits

The methods and kits described herein possess broad applicability in the molecular arts, since many infectious diseases have been linked to specific microorganisms, many of which have known type- or species-specific biomarkers, which may be detected in accordance with the methods described herein. Exemplary pathogens suitable for detection using the disclosed methods include, but are not limited to, bacterial pathogens, viral pathogens, fungal pathogens, unicellular eukaryotic pathogens such as protozoans, spirochetes, prions, or other pathogenic microbiological organisms. Specific examples include, without limitation, the detection of viral pathogens such as HSV, HIV, West Nile Virus, hantavirus, Hepatitis A, Hepatitis B, Norovirus, poliovirus, Rotavirus, etc., the detection of bacterial pathogens such as the causal agents of pneumonia, Legionnaire's disease, food poisoning, food infection, food intoxication, diphtheria, Lyme disease, and/or the detection of protozoal pathogens, including, without limitation, those of the genus Plasmodium.

Kits including one or more of the disclosed pathogen-specific biomarkers or pharmaceutical formulations including such; and instructions for using the kit in a diagnostic, therapeutic, prophylactic, and/or other clinical embodiment(s) also represent preferred aspects of the present disclosure. As used herein, the term “kit” may be used to describe variations of the portable, self-contained enclosure that includes at least one set of reagents, components, or pharmaceutically-formulated compositions to conduct one or more of the diagnostic methods of the invention. Such kits may include one or more of the disclosed pathogen-specific biomarkers, either alone, or in combination with one or more additional diagnostic compounds, pharmaceuticals, and such like. The kits according to the invention may be packaged for commercial distribution, and may further optionally include one or more delivery, storage, or assay components.

The container(s) for such kits may typically include at least one vial, test tube, flask, bottle, syringe or other container, into which the pathogen-specific biomarker composition(s) may be placed. Alternatively, a plurality of distinct biomarker composition(s) and/or distinct proteolytic enzymes may be prepared in a single formulation, and may be packaged in a single container, vial, flask, syringe, catheter, cannula, bottle, test tube, ampoule, or other suitable container. The kit may also include a larger container, such as a case, that includes the containers noted above, along with other equipment, instructions, and the like.

Such kits can, of course, be part of a larger system. For example, the system can also include an instrument such as, for example, a mass spectrometry device for detecting and/or quantitating a target analyte. Sample preparation items can also be included in the various systems and kits, as well as specific instructions for obtaining the samples, processing them, and/or performing one or more steps of the methods disclosed herein.

By way of example, a kit for use involving detection of pathogen-specific biomarkers using a plurality of energy-mediating nanoparticles can be provided that includes two or more of the following:
a population of energy-mediating nanoparticles;
a least one fragment of a biomarker-specific target protein, peptide, or antigen, having at least one mass fingerprint that is detectable by mass spectrometry,
an elution buffer adapted for extracting the at least one fragment of the target protein from the plurality of energy-mediating nanoparticles,
a washing buffer adapted for washing the plurality of energy-mediating nanoparticles before a digestion buffer is added, and
instructions for using the kit to detect a disease biomarker-specific target protein, peptide, or antigen.

Various further changes and modifications to the embodiment herein chosen for purposes of illustration will readily occur to those skilled in the art. To the extent that such modifications and variations do not depart from the spirit of the invention, they are intended to be included within the scope thereof. Accordingly, it is intended that the invention not be limited to the described embodiments, but will have full scope defined by the language of the following claims.

### EXAMPLES

### Example 1: Detection of Peptides of ESAT-6 and CFP-10

Within an Mtb diagnostic assay, peptides from ESAT-6 and CFP-10 were detected simultaneously from human body fluids. First, the finger printing spectra of ESAT-6 and CFP-10 were established. Two major digestion fragments of ESAT-6 (molecular weights 1900.9511 and 1907.9246) were observed in the spectrum. Compared to ESAT-6 fragments, more CFP-10 fragments were observed in MALDI-TOF MS, including peptides of 2003.9781, 1668.7170, 1593.7503, 1142.6276, and 908.4584 Da (Table 1). The most significant CFP-10 fragment (2003.9781) showed 10-fold higher in the intensity than the ESAT-6 fragment, which improved the sensitivity of the assay.

TABLE 1
Amino Acid Sequences and MWs of
ESAT-6, CFP-10 and VP40 Digested Fragments

| | [M + H]+ | Amino Acid Sequence | SEQ ID NO: |
|---|---|---|---|
| ESAT-6 | | | |
| | 1900.9511 | WDATATELNNALQNLAR | (SEQ ID NO: 1) |
| | 1907.9246 | LAAAWGGSGSEAYQGVQQK | (SEQ ID NO: 2) |
| CFP-10 | | | |
| | 2003.9781 | TQIDQVESTAGSLQGQWR | (SEQ ID NO: 3) |
| | 1668.7170 | ADEEQQQALSSQMGF | (SEQ ID NO: 4) |
| | 1593.7503 | TDAATLAQEAGNFER | (SEQ ID NO: 5) |
| | 1142.6276 | GAAGTAAQAAVVR | (SEQ ID NO: 6) |
| | 908.4584 | QAGVQYSR | (SEQ ID NO: 7) |
| VP40 | 1100.21 | QPLPAATWTD | (SEQ ID NO: 8) |

### Example 2: Energy-Mediating Particle-Based Detection Assays

Although CFP-10 and ESAT-6 are abundantly secreted by Mtb, only small amounts of the proteins are released in the peripheral blood of infected individuals. To quantitate these low-abundance biomarkers from blood samples, an assay was developed using ENPs in accordance with the present invention combined MALDI-TOF MS to identify and quantitate pathogen-specific biomarkers from patient samples, including, for example, human sera. This assay is referred to as ENP-MS.

The ENP-MS assay may employ a one-step, fast, microwave-assisted digestion to efficiently digest serum proteomic biomarkers (such as the Mtb-specific peptides, CFP-10 and ESAT-6) into smaller fragments that can be purified, enriched, and specifically detected and quantitated using MALDI-TOF MS. By introducing customized, peptide-specific antibody-ENP conjugates into the digested serum samples, target CFP-10- and ESAT-6-derived peptides have been detected and quantitated using the methods in accordance with the present invention.

The clinical utility of the ENP-MS assay in identifying active TB cases, and the evaluation of treatment efficacy with multiplexed clinical sample cohorts across different countries are demonstrated with the current assay.

### Microwave-Assisted Tryptic Digestion of Human Serum Samples.

Sequencing-grade, modified trypsin (Promega) was resuspended with resuspension buffer (Promega, Madison, Wis.) to 1 mg/mL. For each human serum sample, 100 of the sample was mixed with 400 μL of 100 mM NH₄HCO₃ solution and 10 μL trypsin solution in a 1.5 mL centrifuge tube (Eppendorf, Hauppauge, N.Y.). Sample tubes were placed in a container with 1000 mL of water as a water bath to absorb the extra microwave energy. Samples were digested in a 1200 W household microwave oven (Panasonic, Lake Forest, Calif.) operating at 20% power for 20 min. Fresh water was changed every 10 min in the water bath. After the microwave irradiation, the digestion was terminated by adding trifluoroacetic acid (TFA) to each sample to a final concentration of 0.1%.

### Characterization of ENPs.

The morphology of ENPs was acquired using a Zeiss Neon 40 scanning electron microscope at 5 kV acceleration voltage. The surface oxidation of ENPs was imaged using high resolution TEM (JEOL 2100, 200 kV, Peabody, Mass.). The elemental X-ray analysis maps were generated using scanning TEM with an energy-dispersive X-ray spectroscopy (EDS) detector (Peabody, Mass.).

### Functionalization of ENPs.

Bare ENPs were fabricated and stored in isopropyl alcohol (IPA) for maximum stability. To functionalize ENPs, approximately 7 billion ENPs in 50 mL IPA were transferred to each centrifuge tube. Each tube was centrifuged at 4000 rcf for 20 min to separate the IPA and ENPs, followed by drying under vacuum to completely remove the IPA. 3 mL DMSO was added to each tube, and incubated for 1 hr with rotation to oxidize the surface of the ENPs to silica. DMSO was then removed by centrifugation (4000 rcf, 20 min) and ENPs were washed 3 times with ethanol. (3-Glycidyloxypropyl)trimethoxysilane (GLYMO, Sigma-Aldrich, St. Louis, Mo.) was used to functionalize the oxidized ENPs. GLYMO solution was prepared by mixing 3.5 mL ethanol, 3.5 mL DI water and 3 mL GLYMO. The solution was activated by adjusting the pH to 3.5 using HCl before its addition to the ENPs. After incubating 3 hr in GLYMO solution with rotation, ENPs were separated by centrifugation (4000 rcf, 20 min) and washed three times with ethanol. The resulting functionalized ENPs were resuspended in 10 mL acetone for subsequent use.

### Antibody Immobilization.

Each customized peptide-specific antibody (GL Biochem, Shanghai, China) was immobilized on the ENPs by the direct linkage between the amino group of the antibody and the epoxide group of GLYMO. Briefly, 1 mL ENP solution from the aforementioned stock was centrifuged (10000 rcf, 5 min) in a 1.5-mL centrifuge tube (Eppendorf) followed by drying under vacuum in order to completely remove the acetone (rcf=relative centripetal force). The dry ENPs were resuspended in 1 mL 1× phosphate buffered saline (PBS) (pH=8.6) containing 20 μg antibody (anti-1593.75 or anti-1900.95), and incubated for 2 hr with rotation. The antibody-conjugated ENPs were then separated from the antibody solution by centrifugation (10000 rcf, 5 min) and resuspended in 1 mL 200 mM Tris+100 mM NaCl solution (pH=7) for a 30-min incubation to deactivate the free epoxide groups. Finally, the antibody-conjugated ENPs were washed three times with 1×PBS (pH=7.4) by centrifugation (10000 rcf, 5 min) and resuspension. The resulting antibody conjugated ENPs were resuspended in 60 μL 1×PBS (pH=7.4) for subsequent use.

### Simultaneous Direct Capture of CFP-10 and ESAT-6 Fragments.

10 nM of isotope-labeled 1593.75 fragment (1603.60) and 10 nM of isotope-labeled 1900.95 fragment (1910.80) were added to each tryptic-digested human serum sample for quantification purposes. Next, 10 μL of anti-1593.75-conjugated ENPs and 10 μL of anti-1900.95-conjugated ENPs were added to each tryptic-digested human serum sample, and incubated for 2 hr with rotation in order to capture CFP-10 and ESAT-6 fragments. The conjugated ENPs were then separated from the human serum sample by centrifugation (10000 rcf, 5 min) and washed three times with 1 mg/mL 1,2-dioleoyl-sn-glycero-3-phospho-L-serine (DOPS, Avanti Polar Lipids, Alabaster, Ala.) water solution by centrifugation (10000 rcf, 5 min) and resuspension. The resulting conjugated ENPs were resuspended in 6 μL deionized water for MALDI-TOF-MS analysis.

### MALDI-TOF-MS Analysis.

1.5 μL of each sample was spotted on the target plate and allowed to dry completely under vacuum. Next, 1.5 μL of matrix solution (4 g/L of α-cyano-4-hydroxycinnamic acid (CHCA) in 50% ACN and 0.1% TFA) was spotted on each sample spot and allowed to dry completely under vacuum. MALDI-TOF mass spectra were acquired in the positive reflection mode in the range of 1000-3000 Da. Low mass deflection was set at 800 Da. All tests were performed under high vacuum at or below 10⁻⁷ Torr. The acceleration voltage, electron voltage, and lens voltage were 19 kV, 20 kV, and 9.75 kV, respectively, with a delay time of 100 ns. Mass spectra were acquired from 3000 laser shots for each sample. All spectra were externally calibrated using a peptide calibration standard and raw spectra data were processed with flexAnalysis 3.0 (Bruker Daltonics, Billerica, Mass.).

### Example 3: Detection of TB Using Compositions of the Invention

### Assay Performance Characteristics.

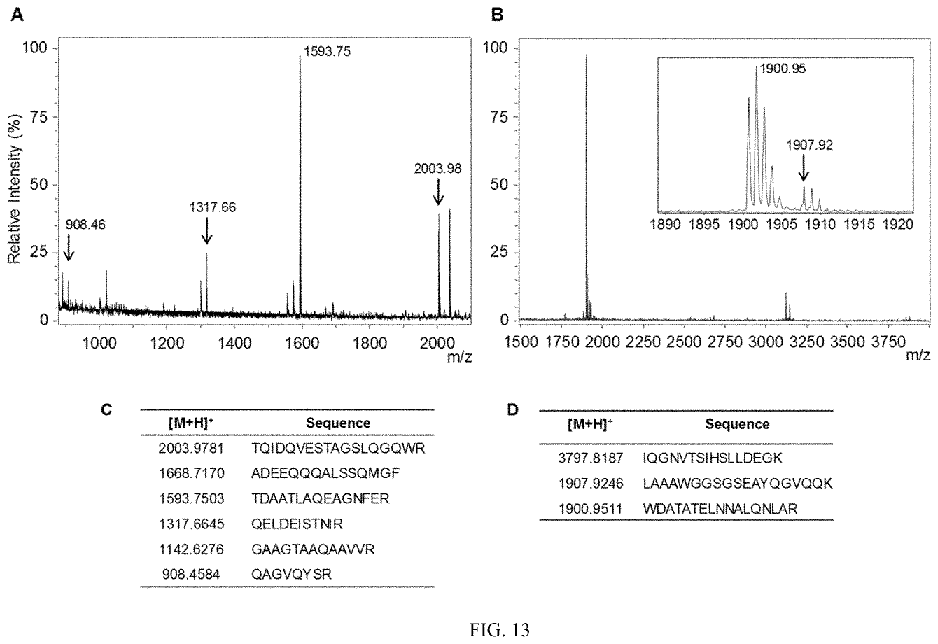

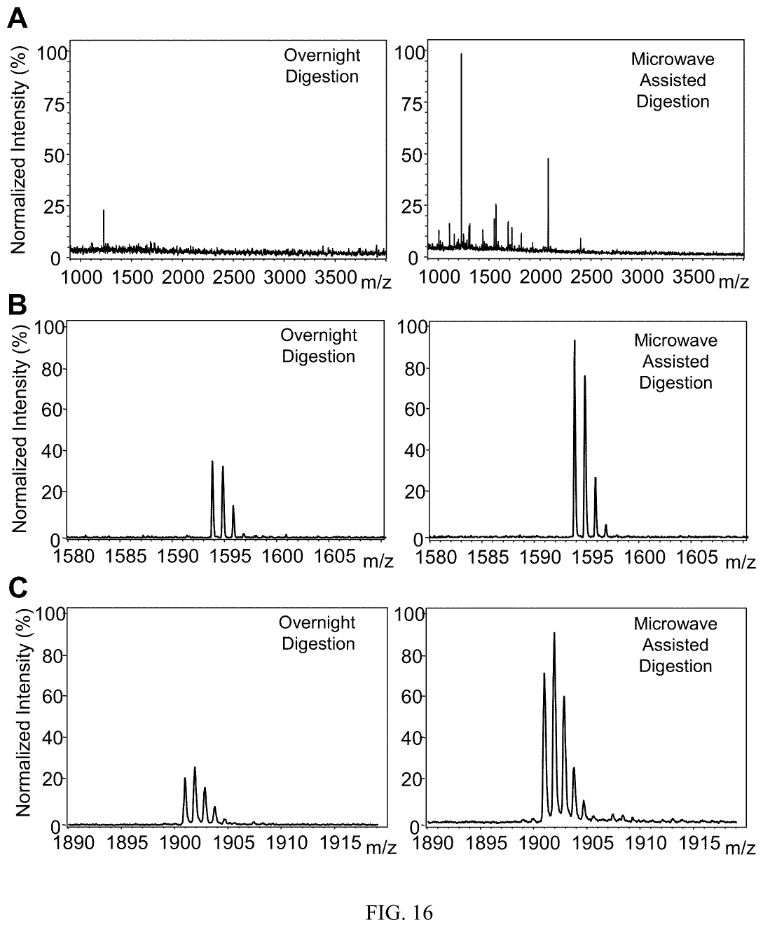

To identify the suitable CFP-10 target peptide and ESAT-6 target peptide for the detection on MALDI-TOF MS platform, CFP-10 (recombinant) and ESAT-6 (recombinant) were digested using trypsin and analyzed using MALDI-TOF MS. CFP-10 peptide 1593.75 and ESAT-6 peptide 1900.95 were selected as representative target peptides due to their high signal-to-noise ratio on the resulting spectra (FIG. 13). Traditional tryptic digestion lacks efficiency in the digestion of complex human bodily fluid such as serum. To overcome this obstacle, tryptic digestion was performed using microwave irradiation to accelerate protein hydrolysis. It was found that the efficiency of peptide yield from microwave-assisted digestion was significantly better than the standard overnight digestion alone (FIGS. 16A-C; (A) MALDI-TOF MS spectra of healthy serum spiked with CFP-10 and ESAT-6 after traditional overnight or microwave assisted trypsin digestion and desalting. Corresponding zoomed-in spectra showing (B) CFP-10 target peptide 1593.75 and (C) ESAT-6 target peptide 1900.95). Target peptide (1593.75 and 1900.95) yields were increased ˜20- and ˜10-fold, respectively when microwave-assisted digestion was employed (FIGS. 16A-C). Furthermore, microwave-assisted digestion provided shorter sample-to-answer time by reducing the digestion time from overnight to 20 min.

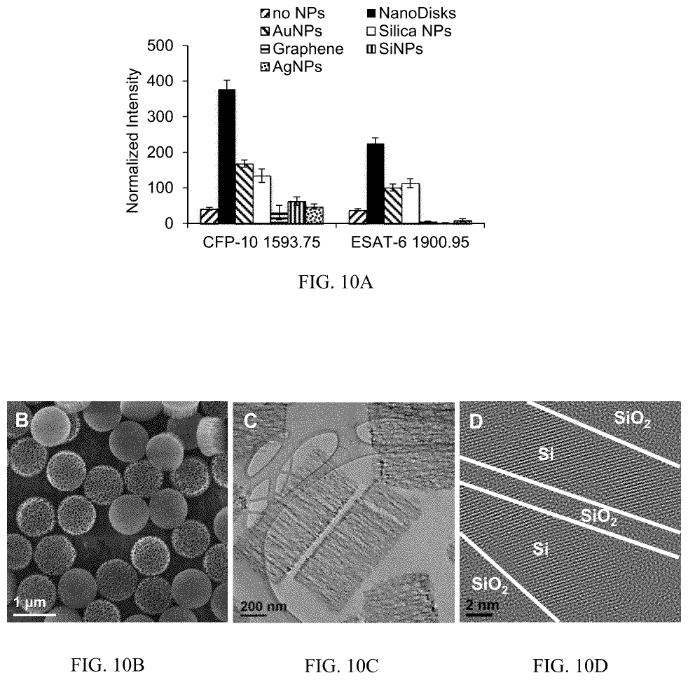

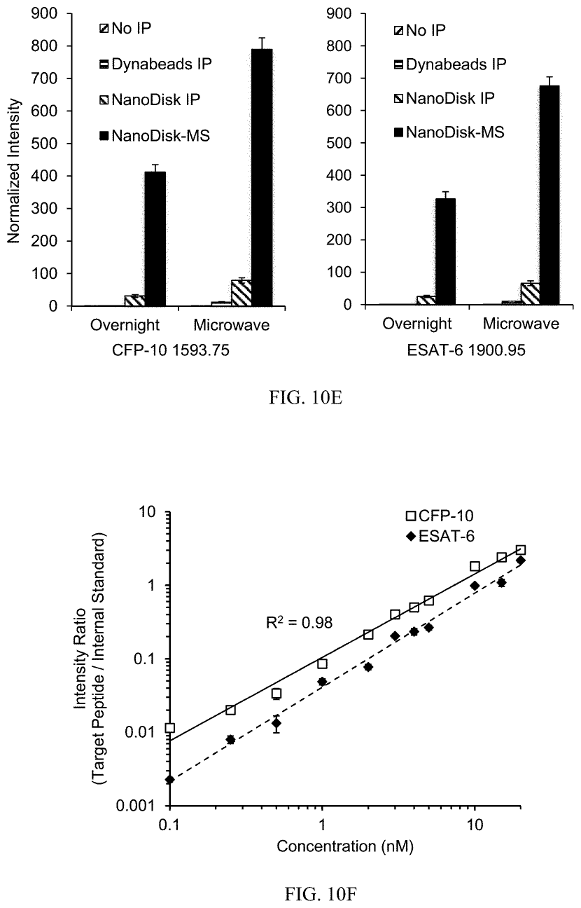

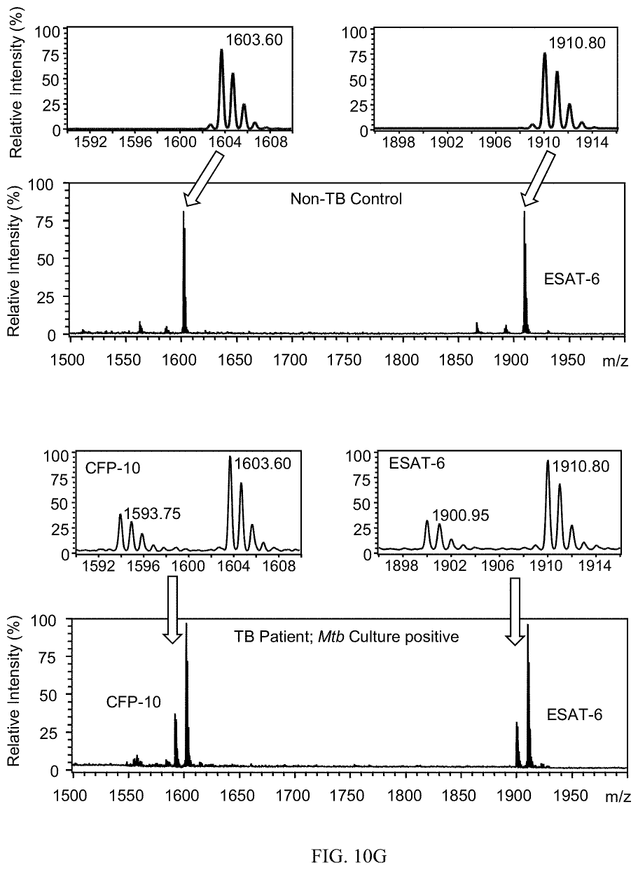

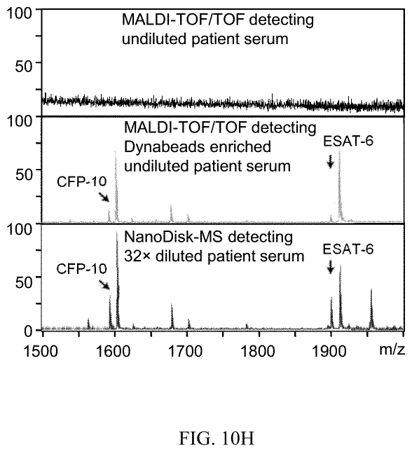

ENP nanotechnology was exploited to overcome the major limitation of low signal intensity in the detection of low-abundant biomarkers using MALDI-TOF MS. MS analysis was performed for the two target peptides, and the signal intensities were compared to that of the target peptide mixed with ENPs. Because of its high absorptivity in the ultraviolet range, ENPs act as energy receptacles for the laser radiation, and hence augment vaporization and ionization of the target peptides. MALDI-TOF MS results showed that the signal intensity was enhanced by ˜9-fold and ˜6-fold for peptide 1593.75 and peptide 1900.95, respectively (FIG. 10A). A calibration curve for peptide 1593.75 and peptide 1900.95 was first established using ENPs mixed with samples containing known concentrations of pre-digested CFP-10 and ESAT-6 proteins, which demonstrated the high reproducibility and linearity of the novel ENP-MS platform (FIG. 10F).

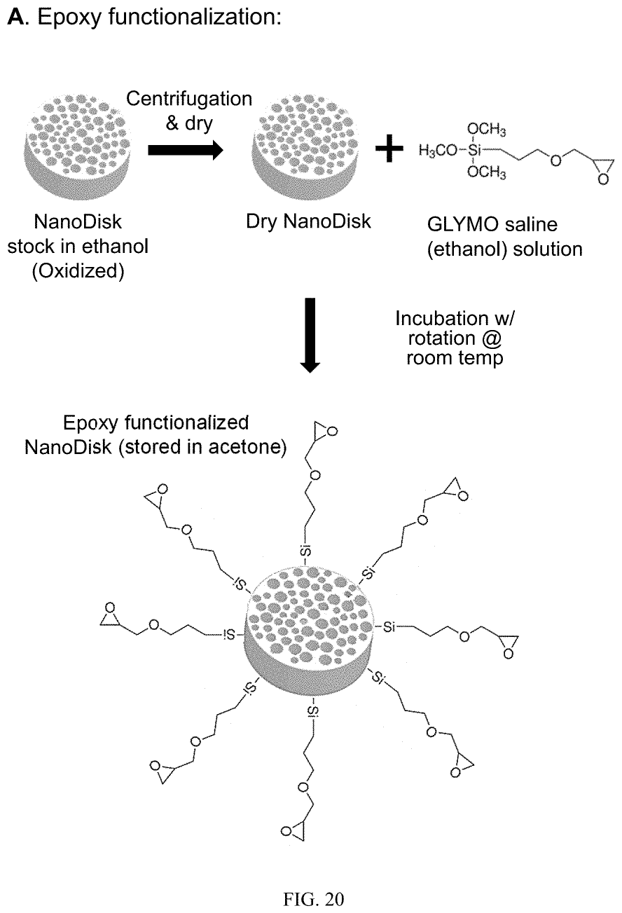

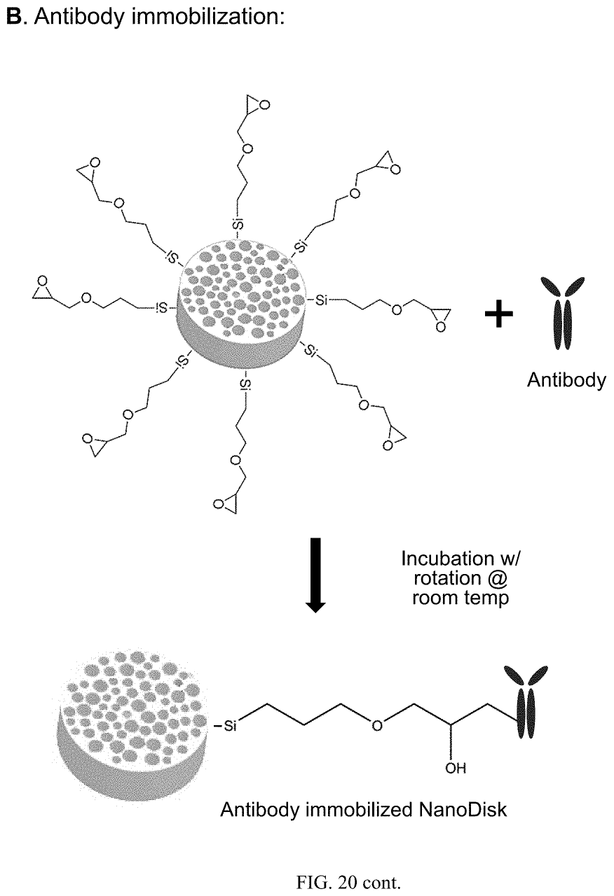

In addition to enhancing MALDI signal intensity and improving sensitivity, ENPs also served as a substrate for purifying and enriching the target peptides. To specifically recognize target peptides 1593.75 and 1900.95, two customized peptide-specific antibodies (anti-1593.75 and anti-1900.95) were obtained. The ENPs were first oxidized using dimethyl sulfoxide (DMSO), then scanning electron microscopy (SEM) and transmission electron microscopy (TEM) were performed to characterize the surface of the ENPs. SEM and TEM images confirmed the reproducibility of 1000-nm (diam.)×400-nm ENPs with 40-nm average pore diameters (FIGS. 10B, 10C and 18A). After oxidation, the silica thin layer on both the inner pore structure and the outer surface of ENPs were observed using TEM (FIGS. 10D and 18B). Finally, the silica surface of ENPs were functionalized with epoxy groups and immobilized with target peptides antibodies (anti-1593.75 and anti-1900.95) (FIG. 20).

### Multiplex Detection and Quantification of Target Biomarkers in Human Serum.

The ENP-MS platform was next used to simultaneously detect the levels of CFP10 and ESAT-6 proteins in human serum samples. CFP-10 and ESAT-6 are known to form both hetero- and homo-dimers naturally, hence their expression levels may be different within the same subject. A positive, active TB case can be diagnosed if one or both of the biomarkers is detected in a sample taken from a patient suspected of infection.

To validate the ENP-MS platform for ultrafast TB diagnosis, volunteer patients with clinically-confirmed, active TB were tested, as well as volunteer patients with LTBI, and healthy volunteers as TB-negative controls. For each subject, 100 μL of human serum was digested and processed with ENPs. Sequencing-grade modified trypsin (Promega) was dissolved with resuspension buffer (Promega) to 1 mg/mL. For each human serum sample, 100 μL of the sample was mixed with 400 μL of 100 mM NH4HCO3 solution and 10 μL trypsin solution in a 1.5 mL centrifuge tube (Eppendorf). Sample tubes were placed in a container with 1000 mL of water as a water bath to absorb the extra microwave energy. Samples were digested in a 1200 W household microwave oven (Panasonic), operating at 20% power for 20 min. After microwave irradiation, each sample digestion was stopped by the addition of trifluoroacetic acid (TFA) (0.1% final concentration).

Following MALDI-TOF MS analyses, it was possible to differentiate active TB cases from LTBI cases; moreover, none of the healthy volunteer control samples were reactive. Peak signals at 1593.75 and/or 1900.95 on mass spectra indicated that peptide 1593.75 (CFP-10) and/or peptide 1900.95 (ESAT-6) was present in the TB-infected samples (FIG. 10G). To quantify the target peptides, isotope-labeled peptides 1593.75 and 1900.95 were introduced into the human serum samples as internal standards for the respective target peptide. The signal intensities of peaks 1593.75 and 1900.95 were normalized to those of the internal standards (MWs 1603.60 and 1910.80). The ratio of the original peak intensity to the internal standard peak intensity was then used for further quantification. Calibration curves for CFP-10 and ESAT-6 were obtained by preparing controls that consisted of human serum samples spiked with known concentrations of CFP-10 and/or ESAT-6 proteins, and then processed and assayed using the same methods as employed for the test samples. A linear correlation between the peak ratio and biomarker concentration was established for each peptide (FIG. 10F). Notably, the calibration curves exhibited >0.98 coefficient-of-determination among different preparations of ENPs, at a detection limit of 0.1 nM and 0.5 nM for CFP-10, and ESAT-6, respectively.

### Example 4: Nanoparticle-Based Assays to Monitor Treatment Efficacy

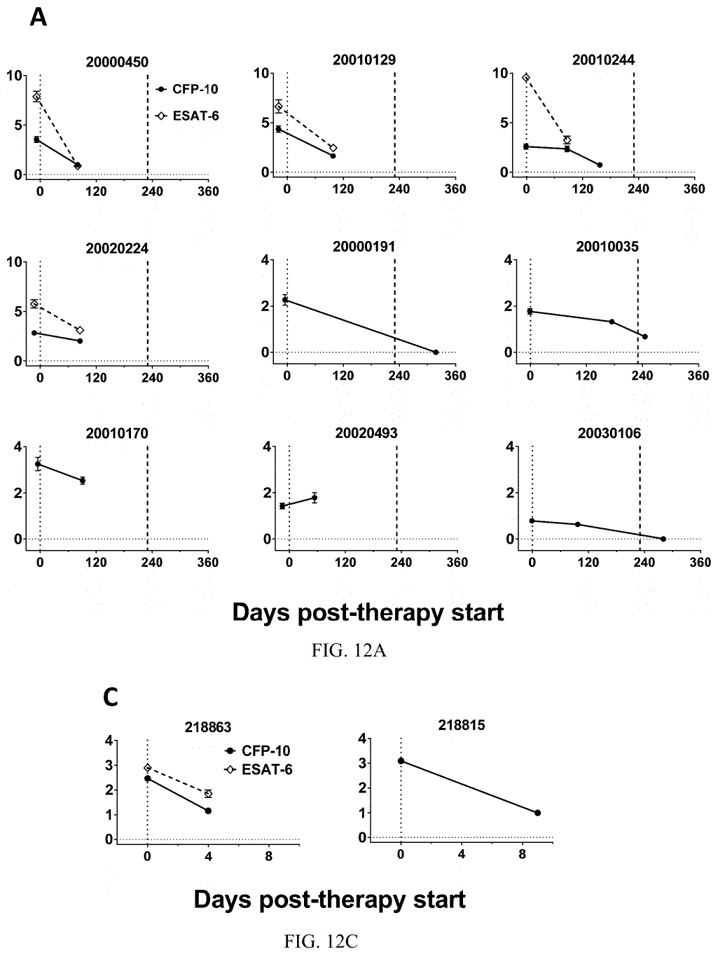

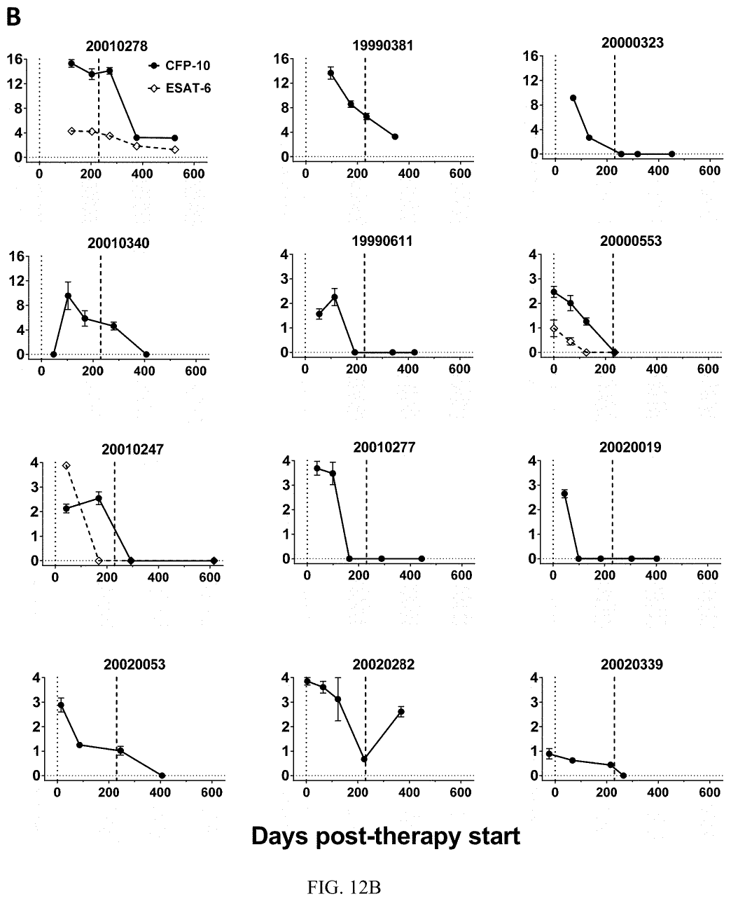

The present invention provides a novel process for monitoring the fluctuations of pathogen-specific biomarkers (such as CFP-10 and ESAT-6) over time as an indication of the effectiveness of particular TB treatment modalities and/or therapies. To demonstrate the facility of the present methods in monitoring therapeutic efficacy, 21 active TB patients with full medical record were followed over time (FIGS. 12A-12C), with blood samples collected prior to, and after initiation of their treatment regimens.

Clinical diagnosis of active TB often involves multiple tests and evaluation from clinical symptoms and contact history. Despite these efforts, TB diagnosis still suffers from low sensitivity and specificity, lack of the ability to distinguish LTBI and long turn-around time, etc. Using the multiplexed TB-specific biomarker detection and quantification platform described above, ongoing evaluation of the treatment of active TB patients has been effectively demonstrated.

Most diagnostic technologies for TB rely on the detection of Mtb bacteria. Currently, the major limitations to these detection technologies are low sensitivity and specificity. Great efforts have been made recently to discover a simple method for active TB diagnosis with improved performance. For example, PCR based GeneXpert™ (Cepheid, Sunnyvale, Calif.) has been reported to provide good specificity, but its moderate sensitivity in extrapulmonary and pediatric patients prevents it from being adopted as a routine TB test. A recently-developed T-cell activation marker assay has enabled TB diagnosis in children. However, measuring T-cell activation marker can lead to false-positives due to the T-cell memory responses in peripheral blood.

When measuring low-abundant proteins such as CFP-10 and ESAT-6 in serum, conventional immunoaffinity-based detection technologies such as ELISA, protein microarrays, quantum dots, and magnetic nanoparticles suffer from high background signal due to non-specific binding with highly-abundant serum proteins. To achieve acceptable specificity, one sacrifices sensitivity to reduce background signals. By integrating an ENP assay with MALDI-TOF MS, however, it is possible to distinguish the signature peaks of CFP-10 and ESAT-6 from peaks of non-specific bound proteins/peptides on the mass spectra, even at the highest sensitivity. By tracking changes in CFP-10 and ESAT-6 expression profiles before and after treatments, physicians should be able to predict and evaluate the efficacy of the therapy, a process that is not possible by existing technologies.

In addition to addressing the current clinical need for improved TB diagnostics, the ENP-MS platform may enable proteomic biomarker detection in a broad range of different diseases. Moreover, this technology can ultimately be deployed to resource-limited areas using portable MS devices for early disease screening and detection.

### Example 5: Quantification of Circulating M. tuberculosis Antigen Peptides Allows Rapid Diagnosis of Active Disease and Treatment Responses

### Sensitive Nanoparticle-Mediated Detection of Mtb-Specific Serum Peptides

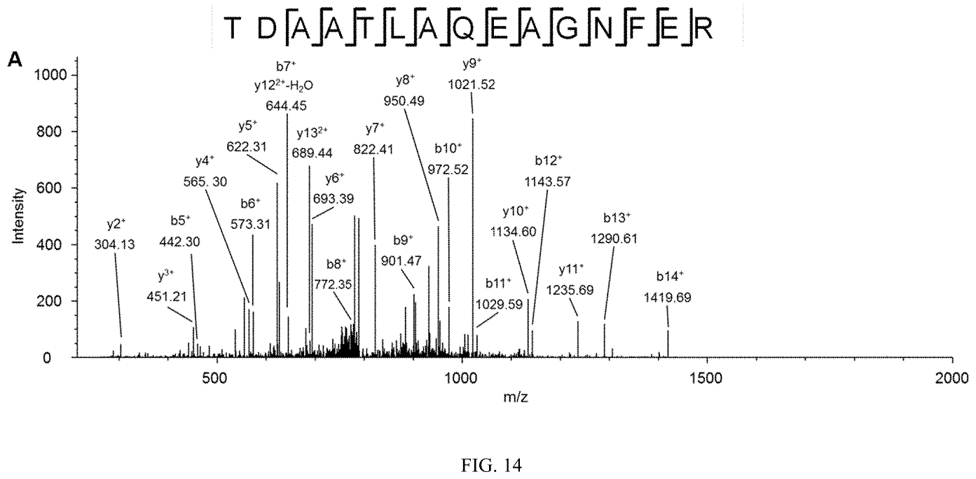

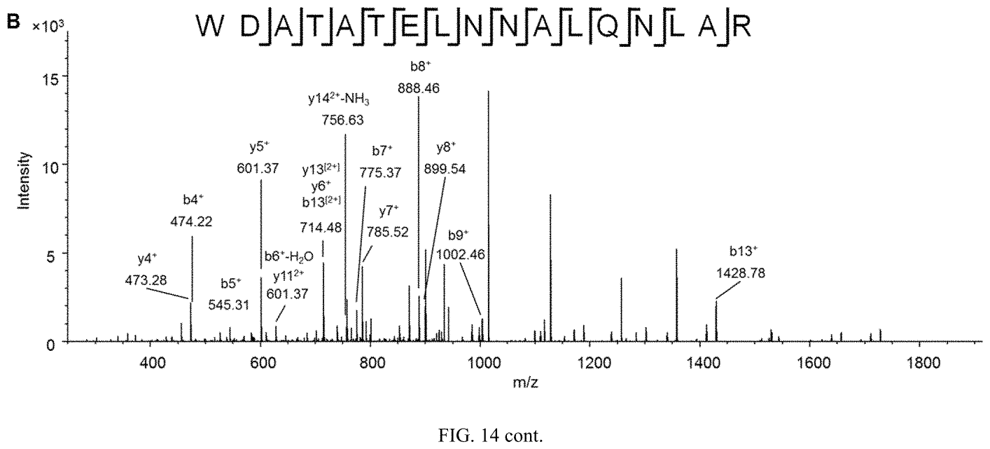

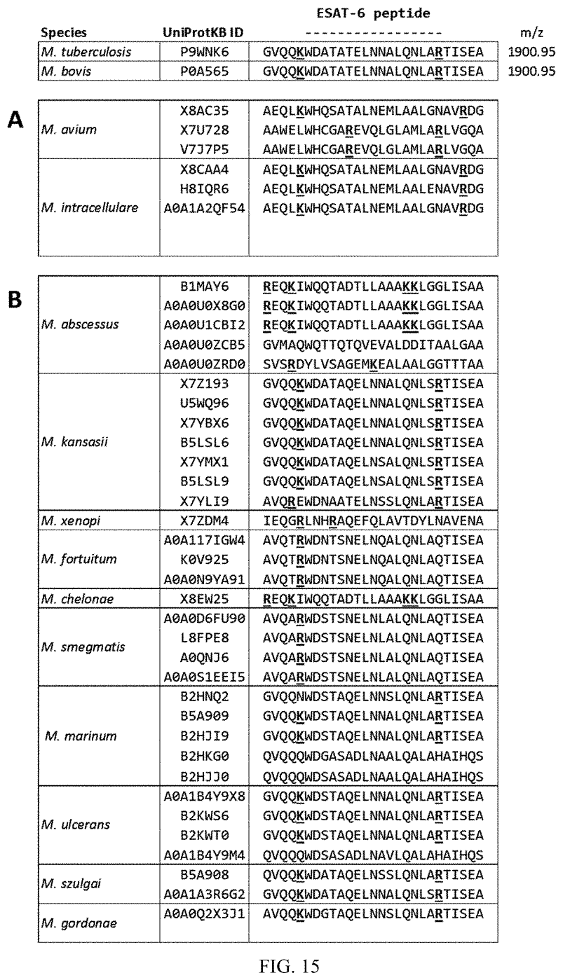

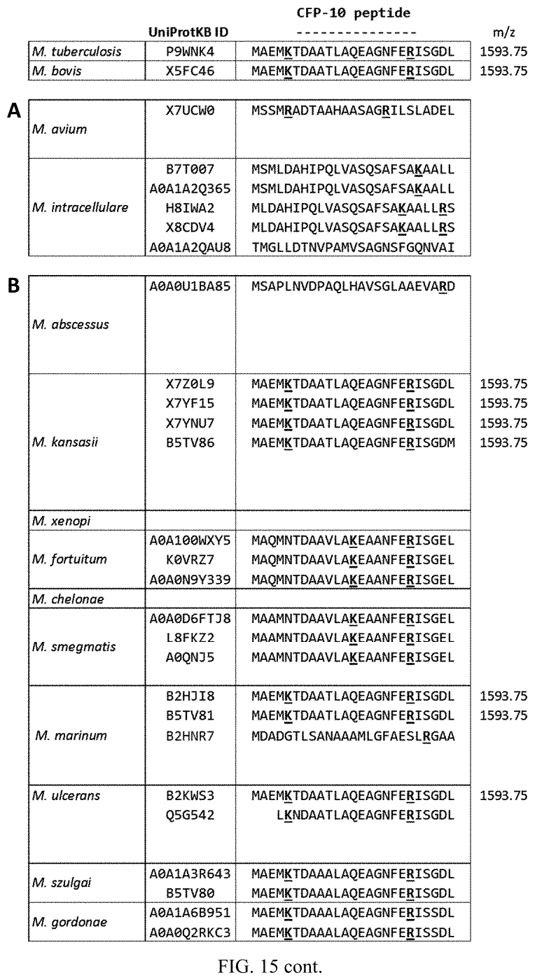

Serum CFP-10 and ESAT-6 expression can theoretically be used to diagnose all active Mtb infections, including EPTB cases; however, some NTM strains express homologues that may reduce the utility of these proteins as biomarkers. Since peptide sequence is the gold standard for protein discrimination, tryptic peptides were examined to distinguish Mtb-derived ESAT-6 and CFP-10 from homologues produced by other species. MALDI-TOF MS analysis of recombinant protein tryptic digests detected CFP-10 (SEQ ID NO. 5; m/z 1593.75) and ESAT-6 (SEQ, ID NO. 1; m/z 1900.95) peptides with high signal-to-noise ratios that were subsequently confirmed by liquid chromatography-tandem mass spectrometry (LC-MS/MS), and which showed strong Mtb specificity when aligned with homologues from 12 NTM species (FIGS. 13-15). Both peptides demonstrated perfect homology with those from M. bovis, a closely related pathogen that causes a relatively rare form of TB, while diverging from two mycobacterium species responsible for the majority of NTM infections, M. avium and M. intracellulare, as well as most other NTM species. The ESAT-6 peptide revealed little homology to any NTM homologue, while the CFP-10 peptide demonstrated full homology only to strains of M. kansasii, M. marinum, and M. ulcerans which were not expected to significantly interfere with diagnostic specificity in clinical usage. See FIG. 15 showing sequence alignment of CFP-10 and ESAT-6 target peptides with corresponding sequence regions in homologues from common NTM clinical isolates of the M. avium complex (MAC) species, which account for the majority of NTM cases, and with other common NTM species. Sequences of Mtb CFP-10 and ESAT-6 and their NTM homologues in the UniProtKB database were aligned by CLUSTAL Omega (1.2.4) multiple sequence alignment (http://www.uniprot.org/align/). The Mtb CFP-10 target sequence TDAATLAQEAGNFER (SEQ ID NO. 5; m/z 1593.75) and ESAT-6 target sequence WDATATELNNALQNLAR (SEQ ID NO. 1; m/z 1900.95) (indicated by a dashed line) are shown with 5 amino acids of adjacent sequence or their N-terminal sequence (CFP-10).

The large dynamic range of serum protein expression can complicate the complete cleavage and subsequent detection on low abundance serum proteins. It was found that supplemental microwave irradiation allowed complete serum CFP-10 and ESAT-6 digestion within 20 min instead of overnight, as normally required for such complex protein samples, reducing the “sample-to-answer” time to four hours while increasing MS signal for target peptides more than 3-fold (FIG. 16).

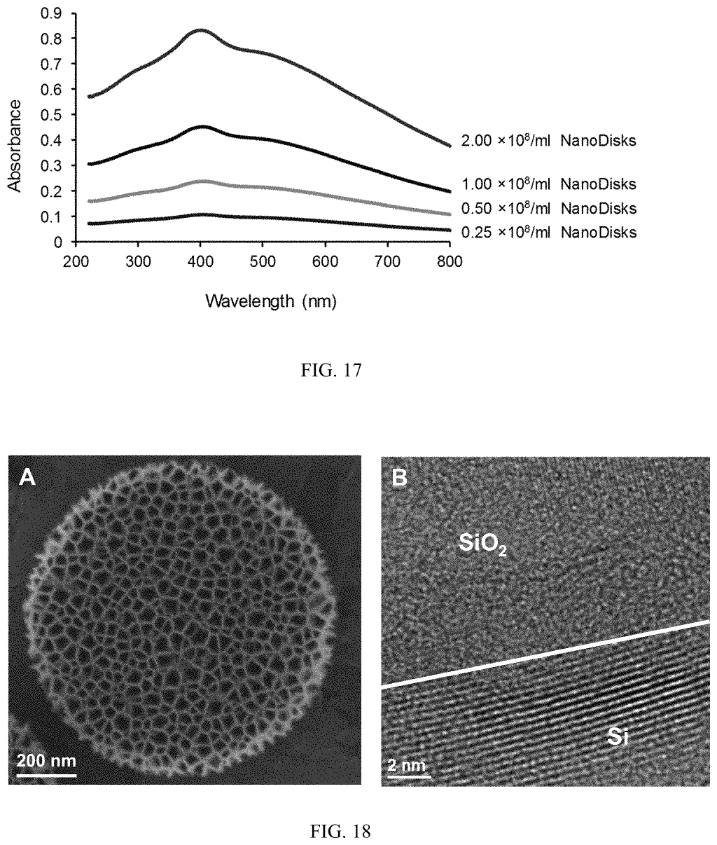

Several potential nanoparticle enrichment platforms were analyzed for their ability to act as MALDI co-matrices, which enhance MS signal by increasing peptide desorption/ionization efficiency. In MALDI-TOF MS analysis of recombinant CFP-10 and ESAT-6 mixed with different nanoparticles respectively, it was found that gold and silica nanoparticles robustly increased MS signal, while graphene and silver and silicon nanoparticles had negligible to negative effects (FIG. 10A). Silicon particles, however, unlike gold and silica particles, can be readily modified to precisely control particle porosity and thus the useful surface area and absorbance properties. A scalable process was developed to rapidly fabricate uniform and inexpensive ENPs with an absorption range that includes the excitation wavelength of the MALDI-TOF MS UV laser (FIG. 17; e.g., NanoDisks exhibit a broad absorbance spectra (λmax=402 nm) that increases with NanoDisk density and overlaps the excitation wavelength (λ=337 nm) of the MALDI-TOF MS UV laser). The surface of ENPs was oxidized to silica for further functionalization. Electron microscopy images of these ENPs revealed highly-reproducible 1000×400 nm discs with 40 nm pores coated with a thin silica layer (FIGS. 10B-D and 18).

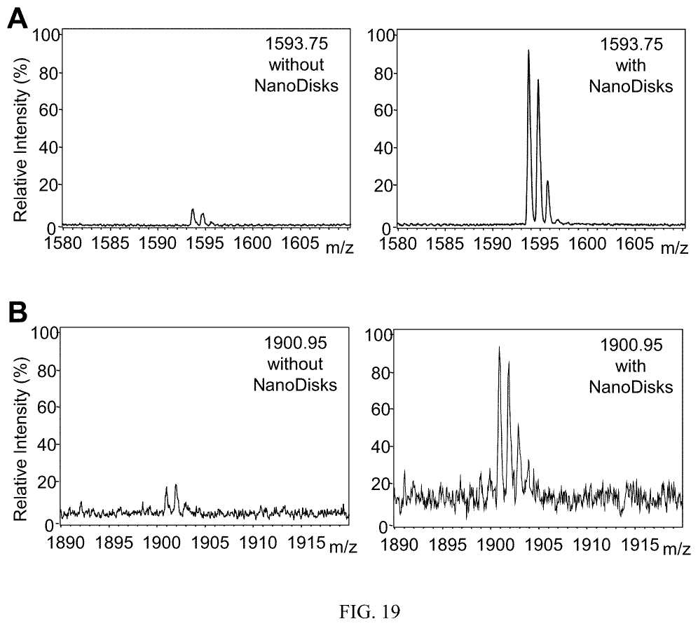

These ENPs demonstrated strongest co-matrix effect (FIGS. 10A and 19), likely due to their UV absorbance properties and thermal confinement effect to promote laser-induced peptide desorption/ionization, and large surface-to-volume ratio that would be expected to trap peptides in close proximity to the co-matrix. The ENPs were then epoxy-modified and conjugated with antibodies specific for the 1593.75 and 1900.95 peptides (FIG. 20) to create a high-affinity, high-capacity peptide enrichment platform. Systematic analysis of MS signal enhancement by microwave digestion, ENP enrichment and co-matrix properties was performed with antigen-spiked healthy human serum, which was split and subjected to overnight or microwave-assisted trypsin digestion followed by MALDI-TOF MS analysis of nonimmunoprecipitated serum, peptides eluted from target-specific Dynabeads or ENPs, and peptides still bound to target-specific ENPs (FIG. 10E). Mtb target peptides were essentially undetectable in overnight or microwave-assisted digests, with or without conventional Dynabeads, but robustly detected when ENPs were used for peptide enrichment or as an enrichment/co-matrix platform. Mean Mtb peptide signal was increased 2.5-fold (CFP-10) and 2.6-fold (ESAT-6) by microwave-assisted digestion, an additional 6.6-fold (CFP-10 and ESAT-6) by ENP-mediated enrichment, and then an additional 9.9-fold (CFP-10) and 10.2-fold (ESAT-6) by ENP co-matrix activity.

### Quantification of Mtb-Specific Peptides in Human Serum

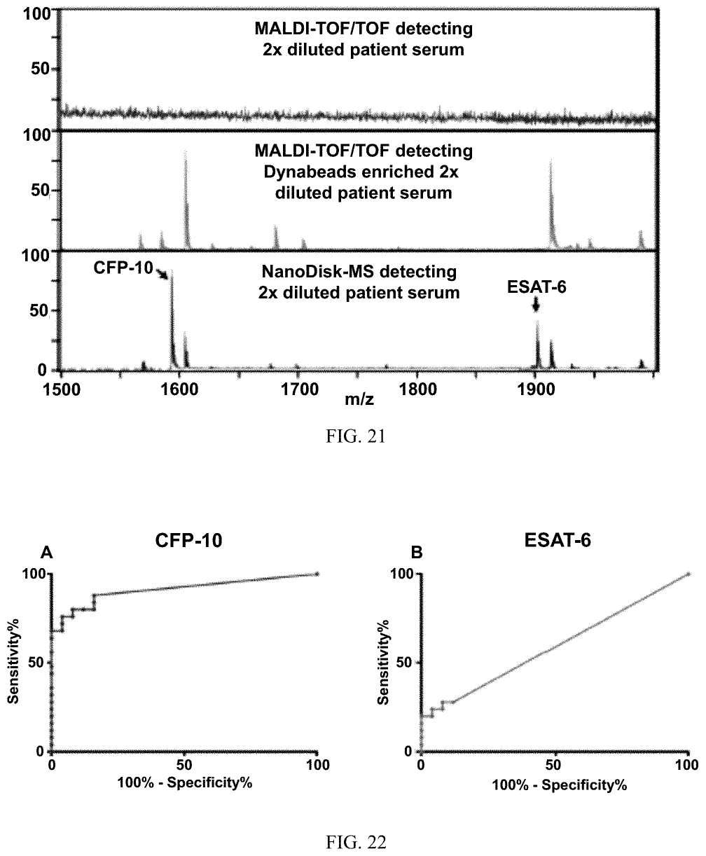

Calibration curves for precise quantitation were generated by digesting recombinant CFP-10 and ESAT-6 standards in TB-free human serum, which were then spiked with stable isotope-labeled internal standards, incubated with target-specific antibody-conjugated ENPs, and then analyzed by MALDI-TOF MS. Excellent correlation (R²>0.98) was observed for curves made with different ENP batches (FIG. 10F), with values exhibiting 14-22% within- and 16-23% between-run coefficients of variation. CFP-10 revealed 50 pM and 200 pM limits of detection (LOD) and quantification (LOQ), while ESAT-6 had a 200 pM LOD and a 500 pM LOQ, with measurement accuracies ranging from ˜74% (1 nM) to ˜90% (20 nM) (Table 2). ENP-MS readily distinguished patients with and without TB (FIG. 10G) in a multiplex assay. Conversely, analyses performed using an advanced MALDI-TOF/TOF MS instrument failed to directly detect target peptides without ENP enrichment in TB patient serum with high CFP-10 and ESAT-6 levels due to the MALDI-inhibitory effects of serum salt and lipid, and only weakly detected target signal after conventional treatment with peptide-specific Dynabeads, which was lost after 2× serum dilution (FIGS. 10H and 21). ENP-MS assays, however, robustly detected both peptides in 2× serially diluted aliquots down to the lowest tested dilution (32×), using either this system (FIGS. 10H and 21) or a more affordable MALDI-TOF MS (not shown).

TABLE 2
Precision and accuracy of CFP-10 and ESAT-6 concentration
measurements (1593.75 and 1900.95 peptides) by ENP-MS

| Concen- | | Precision (% CV) | | |
|---|---|---|---|---|
| tration (nM) | Biomarkers | Within-run | Between-run | Accuracy (%) |
| 20 | 1593.75 | 14.41 | 16.30 | 91.45 |
| | 1900.95 | 17.49 | 18.40 | 89.75 |
| 10 | 1593.75 | 15.07 | 17.29 | 87.90 |
| | 1900.95 | 15.52 | 11.16 | 88.20 |
| 1 | 1593.75 | 19.66 | 15.20 | 78.52 |
| | 1900.95 | 21.49 | 23.36 | 74.30 |

### ENP-MS Diagnostic Sensitivity and Specificity in a HIV-Negative Population

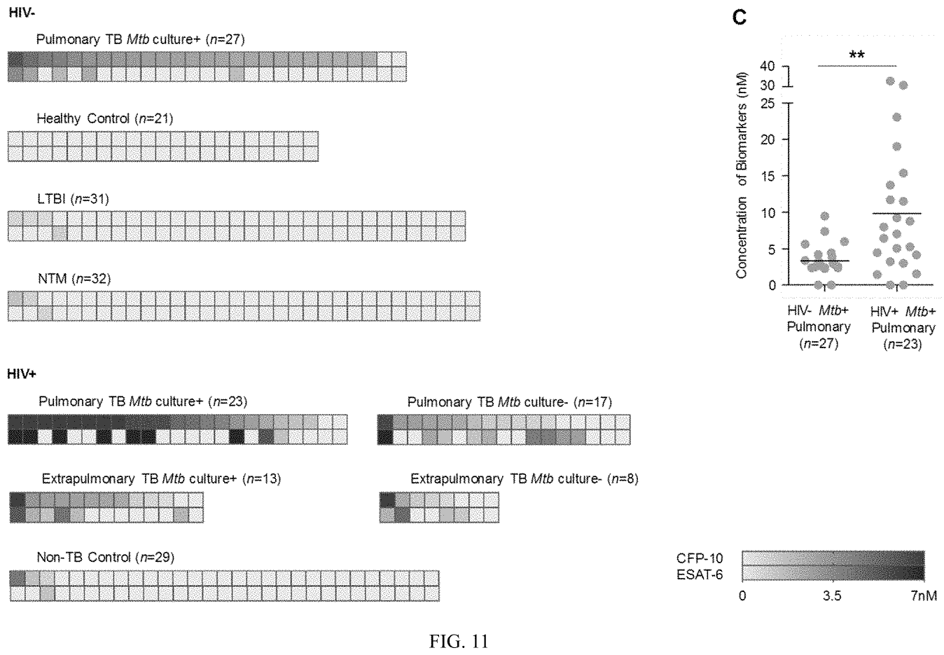

ENP-MS diagnostic performance was assessed with serum of HIV-negative HTI patients, using positive signal of either peptide as the TB diagnostic criteria. Cut-off values of CFP-10 (200 pM) and ESAT-6 (650 pM) concentration were established prior to this study based on the maximum Youden's index in a development cohort including 25 active TB cases and 25 non-TB controls (FIG. 22). Our case-control study contained 27 active PTB, 31 LTBI and 32 NTM cases and 21 healthy controls. Blinded ENP-MS assays detected target peptides in 25 of 27 (92.6%) TB cases (Table 3, FIG. 11), with 100% and 91.0% sensitivities in smear-positive and -negative cases. No target signal was detected in the healthy controls, but false-positive signals were found in 4 of 31 LTBI and 3 of 32 NTM (disease control) patients, for specificities of 87.1% and 90.6%. Notably, LTBI signal may reveal subclinical TB cases, while NTM false-positives may result from detection of relatively rate NTM species (M. kansasii, M. marinum, and M. ulcerans) that, inclusively, account for <5% of NTM cases, since CFP-10 sequence of these strains match the target peptide (FIG. 15B). LBTI follow-up and NTM strain analyses are required to address these questions. However, M. kansasii cases were heavily overrepresented (13 of 32) in the NTM group (Table 4) and 2 of 3 NTM false-positives had M. kansasii infections, suggesting most false-positive could be due to CFP-10 signal from this M. kansasii strain.

TABLE 3
Sensitivity and specificity of ENP-MS for active TB detection.

| | Positive | | |
|---|---|---|---|
| | result no./ | | |
| Group | Total No. | Sensitivity (95% CI) | Specificity (95% CI) |
| HTI cohort (n = 201 adults) | | | |
| HIV− Groups (n = 111) | | | |
| Pulmonary TB Mtb Culture+ | 25/27 | 92.6% (76.6%-97.9%) | |
| LTBI | 4/21 | | 87.1% (71.2%-94.9%) |
| NTM | 3/32 | | 90.5% (75.8%-96.8%) |
| Healthy Controls | 0/21 | | 100% (84.5%-100%) |
| HIV+ Groups (n = 90) | | | |
| Pulmonary TB | | | |
| Mtb Culture+ | 21/23 | 91.3% (73.2%-97.6%) | |
| Mtb Culture− | 14/17 | 82.4% (59.0%-93.8%) | |
| Extrapulmonary | | | |
| Mtb Culture+ | 12/13 | 92.3% (66.7%-98.6%) | |
| Mtb Culture− | 6/8 | 75.0% (40.9%42.9%) | |
| Non-TB | 3/29 | | 89.7% (73.6%-96.4%) |

TABLE 4
Demographics, microbiology and diagnostic data
for studied patients and controls

| | HIV-negative | | HIV-positive | |
|---|---|---|---|---|
| | Cases | Controls | Cases | Controls |
| Variable | n (%) | n (%) | n (%) | n (%) |
| Total number | 27 | 84 | 61 | 29 |
| Males | 22 (81) | 61 (73) | 42 (69) | 22 (76) |
| Age: median (range) | 46 (20-87) | 45 (21-75) | 40 (22-60) | 40 (26-59) |
| Pulmonary | 27 (100) | — | 40 (66) | — |
| Mtb culture positive | 27 (100) | — | 36 (59) | — |
| AFB smear positive | 5 (19) | — | 10 (16) | — |
| PPD positive | — | 31 (37) | — | — |
| NTM | — | 32 (38) | — | — |
| MAC | — | 9 (11) | — | — |
| M. kansasii | — | 13 (15) | — | — |
| M gordonae | — | 1 (1) | — | — |
| M. avium | — | 3 (4) | — | — |
| M. chelonae-abscessus | — | 2 (2) | — | — |
| M. abscessus | — | 3 (4) | — | — |
| M chelonae | — | 1 (1) | — | — |

### ENP-MS Diagnostic Sensitivity and Specificity in a HIV-Positive Population

Non-invasive diagnosis of EPTB patients is challenging due to the paucibacillary nature of their sputum samples, so Mtb cultures often use more invasive specimens, including lymph nodes and pleural or cerebrospinal fluid. EPTB cases are particularly common in HIV/TB co-infected patients, since HIV infection disrupts pulmonary granulomas to reduce the utility of sputum-based diagnostic tests, while altered immune responses in these patients may limit the utility of T-cell-mediated diagnostic assays. Therefore serum samples from HIV-positive HTI patients with culture-positive or -negative PTB or EPTB were analyzed. Blinded analyses identified 91.3% (21 of 23) and 82.4% (14 of 17) of the culture-positive and -negative PTB cases, respectively, and 92.3% (12 of 13) and 75.0% (6 of 8) of the culture-positive and -negative EPTB cases (Table 3 and FIG. 11), while exhibiting 89.7% specificity (26 of 29) for TB-negative/HIV-positive subjects. ENP-MS thus dramatically outperformed Mtb culture-based diagnosis for PTB (57.5%; 23 of 40) and EPTB (61.9%; 13 of 21) cases, and exhibited 100% and 84.3% sensitivities in smear-positive and -negative cases. These results also exceed results from a study published by another group that analyzed Xpert MTB/RIF sensitivity for culture-positive PTB cases (86.2%; 50 of 58) and culture-positive (67.7%; 21 of 31) and negative (29.4%; 5 of 17) EPTB cases in a HIV-positive population.

### Serum CFP-10 and ESAT-6 Levels in HIV-Infected Patients

HIV and TB co-infected patients represent a diagnostically-challenging but demographically important TB population, since HIV-infected individuals are 20 times more likely to develop active TB disease, and estimated to account for 12% of the 9.6 million new TB cases in 2014. Circulating Mtb antigen levels might, however, be increased in these patients, as observed for other bacterial antigens. Indeed, combined CFP+ESAT-6 levels were significantly higher in HIV-positive (9.8 nM) than HIV-negative (3.3 nM) patients with culture-positive PTB cases (FIG. 11). ENP-MS results may permit robust TB diagnoses in HIV-positive patients, since ENP-MS performance exceeds the sensitivity of conventional methods, including front-line alternates such as Xpert MTB/RIF and IGRA.

### Longitudinal Quantification of CFP-10 and ESAT-6 in Patients Under Anti-TB Therapy

Serum Mtb antigen concentrations during anti-TB therapy may reflect therapeutic efficacy. Therefore serial blood samples from 9 HIV-negative and 12 HIV-positive TB patients during and after anti-TB therapy were analyzed, using samples collected during their 6-12 month anti-TB treatment regimen and follow-up. Serum Mtb peptide levels were decreased or undetectable in most HIV-negative (8 of 9) and HIV-positive (11 of 12) TB patients post-therapy (FIGS. 12A and B). The lone non-responsive HIV-negative patient (ID: 20020493) was found to have received an incomplete anti-TB regimen (11 of 20 monthly doses) due to alcohol-induced liver dysfunction and to have exhibited consistent culture-positive results upon review of their health records. One HIV-positive patient (ID: 20020282) revealed CFP-10 decreases that rebounded 2 months after therapy completion, perhaps due to lack of leukocyte bactericidal activity associated with G6PD deficiency, increased infection susceptibility due to hyperferremia from lysis of G6PD-deficient erythrocytes or a decrease in the proportion of CD4+T-lymphocytes. However, most HIV-positive patients who did not achieve Mtb antigen clearance during treatment revealed continued decreases post-treatment and ultimately had undetectable antigen levels, with only one patient demonstrating reduced but relatively stable Mtb antigen levels after treatment completion (ID: 20010278).

Samples from 2 prospectively enrolled patients with active TB prior to and shortly after anti-TB therapy start were also collected. Both patients revealed significant Mtb antigen decreases by 9 days of treatment (FIG. 12C), and were symptom- and culture-negative after 1 month of treatment.

### Example 6: Detection of TB Using Peptides of CFP-10 and ESAT-6 and Compositions of the Invention

Sustained and effective TB control is not a disease management problem exclusive to “third world countries,” since lack of effective vaccines, emergence of drug-resistant TB strains, underperforming diagnostic strategies and slow, culture-based therapy evaluation continue to cost millions of lives worldwide. The ENP-MS assay described herein addresses sensitivity and speed issues associated with active TB diagnosis in diagnostically-challenging patient population, and meets several criteria for a WHO-mandated non-invasive TB assay as it: (1) uses a small, non-invasive specimen; (2) does not require bacterial isolation; (3) has high-sensitivity and -specificity for active TB cases in diagnostically challenging cohorts (e.g., extrapulmonary, culture-negative and HIV-infected TB patients), where diagnoses often require multiple tests, including invasive procedures; (4) directly quantifies Mtb antigens for rapid monitoring of anti-TB therapy effects; (5) uses a streamlined process amenable to high-throughput operation in both clinical and research settings; and (6) can be performed on equipment already FDA approved for other diagnostic assays.

ENP-MS demonstrated similar sensitivity for both PTB (87.5%) and clinically-challenging EPTB (85.7%) cases in the studied HIV-positive population, where it equaled or significantly outperformed recent meta-analysis estimates of Xpert MTB/RIF sensitivity to detect EPTB cases using highly-invasive lymph node (84%), pleural fluid (17%), and cerebrospinal fluid (56%) samples and a study that observed 31%, 69% and 66% sensitivity for AFB smear, MGIT culture and Xpert MTB/RIF results. Moreover, results were achieved irrespective of Mtb culture status, and while greater sensitivity was achieved in culture-positive samples, results from culture-negative samples markedly outperformed the WHO-defined 66% optimal sensitivity for new high-priority non-sputum diagnostic tests. ENP-MS also accurately diagnosed active TB cases in patients with HIV co-infection, which can adversely skew results of blood-based host response immunoassays (e.g., IGRA and TAM-TB), where serum Mtb peptide concentrations were found to equal or exceed those found in HIV-negative subjects.

ENP-MS was also able to precisely quantify serum antigen concentrations, a highly desirable feature for monitoring mycobacterial responses to anti-therapy regimens, since assays currently used to monitor treatment response provide qualitative or semi-quantitative results (smear and culture) or exhibit significant latency (Xpert). The ENP-MS platform may also enable multiplex detection of serum Mtb peptide concentrations for robust detection of active TB cases.

### Microwave-Assisted and Overnight Tryptic Digestion of Human Serum Samples

Serum samples (100 μL) were mixed with 400 μL of 100 mM NH4HCO3 and 104, of 1 mg/mL sequencing-grade modified trypsin (Promega) in 1.5 mL Eppendorf tubes, placed in a 1000 mL water bath, irradiated with a 1200 W microwave using 20% power for 20 min, and then mixed with a 0.1% final concentration of trifluoroacetic acid. Overnight trypsin digestions were incubated at 37° C. for 12 hr using the same trypsin amount and buffer conditions.

### Characterization, Functionalization and Antibody Immobilization of ENPs

Bare ENPs were fabricated and stored in ACS grade 99.9% isopropyl alcohol for maximum stability. Morphology was analyzed with a Zeiss Neon 40 scanning electron microscope at an acceleration voltage of 5 kV. Surface oxidation was imaged using high-resolution transmission electron microscope (JEOL 2100, 200 kV). Functionalized ENP suspensions (1 mL) were pelleted for 5 min at 10,000 g, vacuum dried, suspended in 1 mL PBS (pH 8.6) containing 20 μg anti-1593.75 or anti-1900.95 antibody synthesized by GL Biochem (Shanghai, China), and mixed for 2 hr at 25° C., pelleted for 5 min at 10000×g, incubated for 30 min in 1 mL of 200 mM Tris (pH 7)/100 mM NaCl solution, washed 3× with PBS (pH 7.4), pelleted and suspended in 60 μL PBS (pH 7.4) and then stored at 4° C. until use.

### Quantification of Mtb Antigens in Clinical Serum Samples

Standard curves were generated by spiking healthy donor serum with 0-100 nM recombinant CFP-10 or ESAT-6, subjected to microwave-assisted digestion, spiked with 10 nM stable isotope-labeled internal standard peptide (m/z 1603.60 and 1910.80; GenScript USA Inc.), mixed with antibody conjugated ENPs for 2 hr, pelleted 5 min at 10,000 g, washed 3× with 1 mg/mL 1,2-dioleoyl-sn-glycero-3-phospho-L-serine (Avanti polar lipids), suspended in 6 deionized water and 4.5 μL (1.5 μL×3) and analyzed by MALDI-TOF-MS, using the MS intensity ratios of each target peptide and internal standard. Clinical sample MS intensity ratios were converted to absolute molar concentrations by substitution into this calibration curve. Serum samples used to determine antigen-derived MS signal from trypsinized serum with or without Dynabead-mediated peptide enrichment or ENP-MS peptide enrichment and co-matrix activity were made by serially diluting TB patient serum with healthy human serum (2×, 4×, 8×, 16× and 32×).

### Statistical Analyses

GraphPad Prism 7 software (San Diego, Calif.) was used to generate heat maps and to calculate one-way ANOVAs (Analysis of Variance) with Bonferroni's post-test or Kruskal-Wallis one-way ANOVAs with Dunn's post-test as determined by sample distribution and variance. Differences were considered statistically significant at P<0.05. Diagnostic accuracy of the test was evaluated using receiving operating characteristic (ROC) curve. See FIG. 22. The cut-off values were estimated at various sensitivities and specificities and determined at the maximum Youden's index, i.e. sensitivity+specificity−1. Data are presented as mean±SEM unless otherwise stated.

### Characterization, Functionalization and Antibody Immobilization of ENPs

For functionalization, ˜7×10⁹ bare ENPs were centrifuged at 4000×g for 20 min, vacuum dried, suspended in 3 mL DMSO and mixed by rotation for 1 hr at room temperature. Oxidized ENPs were pelleted for 20 min at 4000×g, washed 3× times 5 100% ethanol, and then mixed by rotation for 1 hr at room temperature with (3 Glycidyloxypropyl)trimethoxysilane (GLYMO, Sigma-Aldrich). GLYMO solution was prepared by mixing 3.5 mL ethanol, 3.5 mL de-ionized water, and 3 mL GLYMO, and activated by adjusting its pH to 3.5 with HCl. After 3 hrs GLYMO incubation, functionalized ENPs were pelleted for 20 min at 4000×g, washed 3× with 100% ethanol, and then suspended in 10 mL ACS grade 99.9% acetone until use.

### Dynabeads Assay

For every 10 samples, 200 μL protein G functionalized Dynabeads (ThermoFisher Scientific) were pelleted, washed and resuspended in 300 μL PBS+0.02% Tween (PBST), then supplemented with 100 μg anti-1593.75 or anti-1900.95 antibody and incubated 60 min at 25° C. with rotation. Antibody-conjugated Dynabeads were then washed 3× with 400 μL PBST and aliquoted into 40 μL PBST for a 3 hr incubation with a digested serum sample. Antigen loaded Dynabeads were pelleted, washed 2× with PBS and 1× with deionized water, and then eluted in 6 μL 1% TFA before MALDI-TOF MS analysis.

### MALDI-TOF MS Analysis

For each sample, 1.5 μL of peptide-loaded ENPs was spotted on the target plate and dried in a vacuum desiccator, then 1.5 μL of matrix solution (4 g/L of a-cyano4-hydroxycinnamic acid (CHCA) in 50% acetonitrile and 0.1% trifluoroacetic acid) was pipetted on each sample spot and evaporated under vacuum. MALDI-TOF mass spectra were acquired using a Bruker Microflex instrument in the positive reflector mode in the m/z 1000-3000 range, with low mass deflection set at 800 Da, and all tests were performed at ≥10-7 Torr. The acceleration voltage, electron voltage, and lens voltage were set at 19 kV, 20 kV, and 9.75 kV, respectively, with a delay time of 100 ns. Mass spectra for each sample were acquired from 3000 laser shots, and externally calibrated using a peptide calibration standard, and raw spectra data was processed with flex Analysis 3.0 (Bruker Daltonics). As specified, high-sensitivity MALDI-TOF MS experiments were performed using a Bruker UltrafleXtreme instrument under similar conditions.

### Quantification of Mtb Antigens in Clinical Serum Samples

LOD and LOQ were determined as signal-to-noise ≥3 and ≥10, respectively. Within-run precision was determined by running five replicates at three concentrations (1, 10, and 20 nM, spiked serum samples) in one run. Between-run precision was determined by analyzing the same three samples (frozen aliquots) in five replicates on three different days. Accuracy was determined using the same three sample aliquots and calculated as the ratio of the mean measured value to the true value. Serum CFP-10 and ESAT-6 concentrations were summed in HIV-negative and HIV-positive PTB patients to allow direct comparison of relative Mtb antigen levels among these patient populations.

What is claimed is:

1. A method of detecting a disease in a subject, the method comprising:
a) isolating a disease-specific target peptide from a subject sample, including the steps of:
i) preparing the subject sample in a manner that generates a plurality of peptides, including the disease-specific target peptide;
ii) contacting the prepared subject sample with an energy-mediating nanoparticle, wherein the energy-mediating nanoparticle comprises:
A) one or more inorganic materials; and
B) an antibody conjugated to the outer surface of the energy-mediating nanoparticle, wherein the antibody specifically binds the disease-specific target peptide; and
iii) washing unbound peptides from the energy-mediating nanoparticle;
b) directly introducing the disease-specific target peptide to a mass spectrometer without treatment or removal of the energy-mediating nanoparticle; and
c) detecting the disease-specific target peptide using the mass spectrometer.

2. The method of claim 1, wherein the disease-specific target peptide is associated with a Mycobacterium tuberculosis infection.

3. The method of claim 2, wherein the antibody specifically binds a peptide of CFP-10 or a peptide of ESAT-6.

4. The method of claim 3, wherein the antibody specifically binds an amino acid sequence selected from the group consisting of SEQ ID NOS: 1-7.

5. The method of claim 1 further including the steps of:
d) quantifying the disease-specific target peptide; and
e) evaluating a severity of the disease based in part by the quantified disease-specific target peptide.

6. The method of claim 1, wherein at least one of the one or more inorganic materials acts as a matrix assisted laser desorption/ionization (MALDI) co-matrix and wherein the introducing step comprises conducting MALDI on the treated subject sample while the disease-specific target peptide is bound to the nanoparticle.

7. The method of claim 1, wherein the preparing step is a technique selected from the group consisting of: digestion with a protease, digestion with a chemical agent, exposure to microwave radiation, and a combination thereof.

8. The method of claim 1, wherein the preparing step and contacting step are performed sequentially or simultaneously.

9. The method of claim 1, wherein the subject sample is a blood-based sample comprising whole blood, plasma, or serum.

10. The method of claim 1, wherein the nanoparticle is a sphere having a diameter of about 50 nm to about 5000 nm.

11. The method of claim 1, wherein the nanoparticle is disk-shaped and has a diameter of about 500 nm to about 5000 nm and a height of about 200 nm to about 500 nm.

12. The method of claim 1, wherein the nanoparticle defines a textured surface.

13. The method of claim 1, wherein the one or more materials are selected from the group consisting of silica, gold, a metal oxide or a metal oxide-hydroxide.

14. The method of claim 1, wherein the nanoparticle absorbs light having a wavelength of about 100 nm to about 500 nm.

15. The method of claim 6, wherein the nanoparticle comprises an inner core comprised of a first material and an outer layer comprised of a second material, wherein the second material acts as the MALDI co-matrix.

16. The method of claim 1, wherein prior to step a), the method further comprises the steps of:
d) identifying the disease-specific target peptide;
e) obtaining the antibody that specifically binds the disease-specific target peptide; and
f) conjugating the antibody to the outer surface of the energy-mediating nanoparticle.

17. The method of claim 16 wherein the identifying step includes the steps of:
i) determining whether a pathogen causing the disease secretes a virulence factor;
ii) analyzing an abundance of the virulence factor and/or a structural or metabolic protein of the pathogen;
iii) determining if the abundant factor and/or structural or metabolic protein is conserved across two or more pathogen species, wherein
a) if the abundant factor and/or structural or metabolic protein is not conserved, determining whether the abundant factor and/or structural or metabolic protein contains a pathogen-specific peptide sequence, whereby the abundant factor or structural or metabolic protein is selected as a candidate single-pathogen-specific biomarker peptide, or
b) if the abundant factor and/or structural or metabolic protein is conserved, proceed to step iv);
iv) determining if the conserved abundant factor and/or structural or metabolic protein also includes a variable sequence portion; and
v) determining if the variable sequence portion distinguishes between pathogen species, whereby if the variable sequence portion distinguishes between pathogen species the conserved abundant factor or structural or metabolic protein is selected as a candidate broad-spectrum-pathogen-specific peptide biomarker.
